SEQUENCE LISTING (4 sequences)

SEQ ID NO: 1 - protein, 854 aa, Homo sapiens
  DISC1 protein
MPGGGPQGAPAAAGGGGVSHRAGSRDCLPPAACFRRRRLARRPGYMRSSTGPGIGFLSPAVGTLFRFPGGVSGEESHHSESRARQCGLDSRGLLVRSPVSKSAAAPTVTSVRGTSAHFGIQLRGGTRLPDRLSWPCGPGSAGWQQEFAAMDSSETLDASWEAACSDGARRVRAAGSLPSAELSSNSCSPGCGPEVPPTPPGSHSAFTSSFSFIRLSLGSAGERGEAEGCPPSREAESHCQSPQEMGAKAASLDGPHEDPRCLSRPFSLLATRVSADLAQAARNSSRPERDMHSLPDMDPGSSSSLDPSLAGCGGDGSSGSGDAHSWDTLLRKWEPVLRDCLLRNRRQMEVISLRLKLQKLQEDAVENDDYDKAETLQQRLEDLEQEKISLHFQLPSRQPALSSFLGHLAAQVQAALRRGATQQASGDDTHTPLRMEPRLLEPTAQDSLHVSITRRDWLLQEKQQLQKEIEALQARMFVLEAKDQQLRREIEEQEQQLQWQGCDLTPLVGQLSLGQLQEVSKALQDTLASAGQIPFHAEPPETIRSLQERIKSLNLSLKEITTKVCMSEKFCSTLRKKVNDIETQLPALLEAKMHAISGNHFWTAKDLTEEIRSLTSEREGLEGLLSKLLVLSSRNVKKLGSVKEDYNRLRREVEHQETAYETSVKENTMKYMETLKNKLCSCKCPLLGKVWEADLEACRLLIQSLQLQEARGSLSVEDERQMDDLEGAAPPIPPRLHSEDKRKTPLKVLEEWKTHLIPSLHCAGGEQKEESYILSAELGEKCEDIGKKLLYLEDQLHTAIHSHDEDLIQSLRRELQMVKETLQAMILQLQPAKEAGEREAAASCMTAGVHEAQA

SEQ ID NO: 2 - protein, 852 aa, Mus musculus
  DISC1 protein
MQGGGPRGAPIHSPSHGADSGHGLPPAVAPQRRRLTRRPGYMRSTAGSGIGFLSPAVGMPHPSSAGLTGQQSQHSQSKAGQCGLDPGSHCQASLVGKPFLKSSLVPAVASEGHLHPAQRSMRKRPVHFAVHSKNDSRQSERLTGSFKPGDSGFWQELLSSDSFKSLAPSLDAPWNKGSRGLKTVKPLASPALNGPADIASLPGFQDTFTSSFSFIQLSLGAAGERGEAEGCLPSREAEPLHQRPQEMAAEASSSDRPHGDPRHLWTFSLHAAPGLADLAQVTRSSSRQSECGTVSSSSSDTGFSSQDASSAGGRGDQGGGWADAHGWHTLLREWEPMLQDYLLSNRRQLEVTSLILKLQKCQEKVVEDGDYDTAETLRQRLEELEQEKGRLSWALPSQQPALRSFLGYLAAQIQVALHGATQRAGSDDPEAPLEGQLRTTAQDSLPASITRRDWLIREKQRLQKEIEALQARMSALEAKEKRLSQELEEQEVLLRWPGCDLMALVAQMSPGQLQEVSKALGETLTSANQAPFQVEPPETLRSLRERTKSLNLAVRELTAQVCSGEKLCSSLRRRLSDLDTRLPALLEAKMLALSGSCFSTAKELTEEIWALSSEREGLEMFLGRLLALSSRNSRRLGIVKEDHLRCRQDLALQDAAHKTRMKANTVKCMEVLEGQLSSCRCPLLGRVWKADLETCQLLMQSLQLQEAGSSPHAEDEEQVHSTGEAAQTAALAVPRTPHPEEEKSPLQVLQEWDTHSALSPHCAAGPWKEDSHIVSAEVGEKCEAIGVKLLHLEDQLLGAMYSHDEALFQSLQGELQTVKETLQAMILQLQPTKEAGEASASYPTAGAQETEA

SEQ ID NO: 3 - DNA, 7069 bp, Homo sapiens
  DISC1 mRNA
ggaaggagcaggaggcagcccaggcggagcgggaggagctggcagcggggcgcatgccaggcgggggtcctcagggcgccccagccgccgccggcggcggcggcgtgagccaccgcgcaggcagccgggattgcttaccacctgcagcgtgctttcggaggcggcggctggcacggaggccgggctacatgagaagctcgacagggcctgggatcgggttcctttccccagcagtgggcacactgttccggttcccaggaggggtgtctggcgaggagtcccaccactcggagtccagggccagacagtgtggccttgactcgagaggcctcttggtccggagccctgtttccaagagtgcagcagcccctactgtgacctctgtgagaggaacctcggcgcactttgggattcagctcagaggtggcaccagattgcctgacaggcttagctggccgtgtggccctgggagtgctgggtggcagcaagagtttgcagccatggatagttctgagaccctggacgccagctgggaggcagcctgcagcgatggagcaaggcgtgtccgggcagcaggctctctgccatcagcagagttgagtagcaacagctgcagccctggctgtggccctgaggtccccccaacccctcctggctctcacagtgcctttacctcaagctttagctttattcggctctcgcttggctctgccggggaacgtggagaagcagaaggctgcccaccatccagagaggctgagtcccattgccagagcccccaggagatgggagccaaagctgccagcttggacgggcctcacgaggacccgcgatgtctctctcggcccttcagtctcttggctacacgggtctctgcagacttggcccaggccgcaaggaacagctccaggccagagcgtgacatgcattctttaccagacatggaccctggctcctccagttctctggatccctcactggctggctgtggtggtgatgggagcagcggctcaggggatgcccactcttgggacaccctgctcaggaaatgggagccagtgctgcgggactgcctgctgagaaaccggaggcagatggaggtaatatccttaagattaaaacttcagaaacttcaggaagatgcagttgagaatgatgattatgataaagctgagacgttacaacaaagattagaagacctggaacaagagaaaatcagcctgcactttcaacttccttcaaggcagccagctcttagcagtttcctgggtcacctggcagcacaagtccaggctgccttgcgccgtggggccactcagcaggccagcggagatgacacccacaccccactgagaatggagccgaggctgttggaacccactgctcaggacagcttgcacgtgtccatcacgagacgagactggcttcttcaggaaaagcagcagctacagaaagaaatcgaagctctccaagcaaggatgtttgtgctggaagccaaagatcaacagctgagaagggaaatagaggagcaagagcagcaactccagtggcagggctgcgacctgaccccactggtgggccagctgtccctgggtcagctgcaggaggtcagcaaggccttgcaggacaccctggcctcagccggtcagattcccttccatgcagagccaccggaaaccataaggagcctccaggaaagaataaaatccctcaacttgtcacttaaagaaatcactactaaggtgtgtatgagtgagaaattctgcagcaccctgaggaagaaagttaacgatattgaaacccaactaccagccttgcttgaagccaaaatgcatgccatatcaggaaaccatttctggacggctaaagacctcaccgaggagattagatcattaacatcagagagagaagggctggagggactcctcagcaagctgttggtgttgagttccaggaatgtcaaaaagctgggaagtgttaaagaagattacaacagactgagaagagaagtggagcaccaggagactgcctatgaaacaagtgtgaaggaaaatactatgaagtacatggaaacacttaagaataaactgtgcagctgcaagtgtccactgcttgggaaagtgtgggaagctgacttggaagcttgtcgattgcttatccagagcctacagctccaggaagccaggggaagcctgtctgtagaagatgagaggcagatggatgacttagagggagctgctcctcctattccccccaggctccactccgaggataaaaggaagacccctttgaaggtattggaagaatggaagactcacctcatcccctctctgcactgtgctggaggtgaacagaaagaggaatcttacatcctttctgcagaacttggagaaaagtgtgaagacataggcaagaagctattgtacttggaagatcaacttcacacagcaatccacagtcatgatgaagatctcattcagtctctcaggagggagctccagatggtgaaggaaactctgcaggccatgatcctgcagctccagccagcaaaggaggcgggagaaagagaagctgcagcttcctgcatgacagctggtgtccacgaagcacaagcctgaggagtgacgggatgggggagggaggtgggccaccatgtttggacccggggggctgctcttccctcccccgccatagctaagatgcctgaatcaattacggagatacagagccttgaggtctttcagtggaaaggtggttcatgttcattctcatcagtgtgaaactgaggagtctgcaatttggaatatggagagagagactgatttgctgaatttccttctaaatgtcactcaaaaatttcttttccatgtcattcttgggaatgtcttccacaggatttgagaatagtttcatctcagcccccattagagagaagttggggtgaattctggaaaaatgtctctttttcctgtgccatttgccttctgctgcaacgaaaatatttcctgattcaagattctataaaaaggaaaccaagcataagactctgtcatcatacctgttacacgttcctacaggtgcacaatctaagagagctaattaacctcagagtctggagttaacagcttttcaccttacttctcctgtgatctaatattatcttagaaaaattaatatgcaatttccaaaagatattttggtaagacaacaacctcccagtgatatgccacctttcaattttccttttgtggcaatgattgcatctgaagaaaggatccctgagagtctctgtttcatcaggacattctgaaatttacccacagtgaggctgtggatggatcaggggacctgtataaaatgtttgagcctgttccattttcccgtggaacctgtttcactcaatgccaggcagtgcagcatttaggaaagcagtgcagtactcagtaaggcagtgcagtactcagtaacacaatacagtactcaggcagtgcagtactcagtaagacagtgcagtgctcagtaaggcagtgcaatactcagtaacagtgtagtactcagtaacagtgaagtactcagtaatacagtacagtattcagtaaggcagtgaagtactcagtaatacaatacagtactcagttaggcagtgcagtactcaggaatgcagcacagtactcaggcagtgcaatactcagtgcggtactcagtaacacagtgcagtactcagtaacagtgcagtactcagtaacagtgcagtactcagtaaggcagtgcagtactcagtaacacagtgcagtactcagtaatacagtacagtactcagtaaggcagtatggtactcagtaaagcaatgcaatgctcagtaacacagtgcagtgctcaataaggcagtgcagtgctcagtaaggcagtgaattgcttagtaacacagtgtagtgctcagtaggacagcatagtactcagtaacacagggcagctagtactcagtactataagtactgagtacttatataggcaatgtagtactcagtaaatcagtgcagtactcagtaatgcaagggcatttcaggctcctgctgggctgcttctttggcccagctgggactcctattgagacagctgcaaaacaggctgatttcaattaggcagcacttcccaaagtgcactgaggaaggtggccccaagagaagctctctaaacaaaggagtaccctctctggtcaagtacctttggtaaatacaccataccataatatctgcttggagaaccacaatgcacattagcatattagtctgagagagaacttatagtaaggaaactcacttgattttatctaacctcaaactttccaagtttaatggatcgtgaatttttttcatgtaactcctattcatatcccatagatctagtattgtacagcactgcattctctgaggaagtcccagtccaaactctgatttacatcactttagaaaccacactcacacttttgcagagtgttgagcttaataactacctgccacagattggtaaatttaatccagtggttgttctgtttgtgcttctgttctcatttatgtgtttagggatagtgaggttcctgccttcactaggatccacggatatgagaccatttttgtcatttcctgaagtcacactggcgtttccagaaggcatctggtgctttgctcagccttccatgctgtgcagcacttctgtcctcagtcaaggagatggccatgcttaagccagcaattggctggggtccaggaaacaaagcaaaagcacaatatgtgaatgtgctgattgtgttccctatggctttatctcgagcaaaatacactctacatattttaataataagtataattagcttgttcctggacttcattttcaatgatgaaccaaattcctgaattatttataattgtgtctaaagaaaattatgaactggtcacatggcacttggaatccttgagttaattccagtgaagcaaaacttgggaagagtcaggattggccacattgccaataacaaattcctacttcgacatatgtcttttcaaaaagcctcccagacacaagacatcttaaccgtcactagcccaagtgttttgtattactcagacaccatcatgaaataattctgtgaggtcatgatgtatttgaaaattctgcaagttaataactgccttgaattgtttgaacccgaaataagggttctttggtacctctagtagatagtgtgttcatttccctgctgcaaattttgaagtatttgggcaggtgagtcatgttttaaccacaagccataactcatctgttgtctttgcttggtcttagagtatcattcagaaagtccgctaagggccagcgtgcttcttctggctacacaaccttctcaggacaagcccactgtcttaagccactttgaccctgggagacacaggactgtgtatcctcaatcatactatacagcagtttttgtcaggggaacataaaaatatccaagagaggttagggcttagatttaaaagcatcaaaacaacaacaatggaaatttatgttggcgatagccaagaccacaagcaaaagcacatactggaaatgatgagttagaatctgatttgactgggatgttttatgagaatgtaagtgtgatattatactgtctgccttgctggaatgctggctttcaaatggtcacccatttttctttcactggcctgagttaggacatgctatcagtaatagtcccagttccatccaactttctgaaatttcatttttttttttgagatggagtctctctctgtcacccaagttggagtgcagtggccccgcaatctcggctcactgcaacctctacctcccaggttcaagctattctactgcctcagcctcccaagtagctggggttacaggcatttgccaccgggccctgatgatttttgtatttttagtagagacagggcttcaccatgttggctaggagggtctcaaactcctgacctcaggcgatccacccccacctcggcttcccaaagtgctgagattacaggtgtgagccaccgcacccggccaactttctgaaatttcaaaactgaattgatccttctccaaattagtatatactattggaaacttgtctttccctgcagtaaggctggtttccccaccccagaaacatgtaacggttggtaccatgctaagcccttgccatgctaagccctttacagtcatatcctataatccccatatcaaccttataaggaaggtgtttgtagatgatgcaactgagccttaagaggactaattccctttttctaaggcacagagctggtaaaatgtgaagtaatagtgaacctaacagtcagagacaggcagcatgctcttaactagtgctcttcctaaagttcctttaatgtccttttgagattttgagccatggaacttacttgttcacctggctaagaactcatggccactgtggaaatcttggttagggagtcaaagaaactgagcctggggcaaacgaggcttcccacactgccaggggagcctcactgtgaagtctaggctcagacaggcatcaacaaacctattcaccccaccatcatcctgatctaaccattccccagtcatcccaggaaaaccactcacagcctgacactgggctgactttcttgaagatcctcatccaattggtgtttttcagaagtgttccaatattatgaattctgtgttgtggagaaaagcaaccatgcatttactggtcaatgccttcttgtatatgtaattcaatacttttacttttaatatcctcaccttatctaatctttgaattttgtcatgtaatttattgcttcattaaggttactttttgttatacaaaataaaagctgatatccaaggcatggtgcatcttgatgattttttgtcctttgaagtatggatgatagaaaaatgtatcaggtttattcatctcatctttctgttacaggatgattaattgtacagttacatcacacgaaacatttataataaagtcatgctttagaaaaaaaaaaaa

SEQ ID NO: 4 - DNA, 2597 bp, Mus musculus
  DISC1 mRNA
cagtcaccgccgtccggggcgcagccggccgcacgatgcagggcgggggtccccggggcgctccgatccacagtccgagccacggcgcagacagtgggcatggcttaccgcctgcagtagcccctcagaggcggcggctgacacggagaccaggctacatgagaagcacagcgggttctgggatcgggttcctctctccagcagtgggcatgccacacccgagctcagcagggctgacaggccagcagtcccaacactcacagtccaaggctggacagtgcggacttgaccctgggagccactgccaagcctcactggtgggcaagccttttctcaagagctcccttgtccctgctgtggcctctgagggccacctgcacccagcccagcgctctatgagaaaaagaccagtgcactttgcggttcattccaagaatgacagcagacaatctgagaggctgactgggtcatttaagcctggggacagtgggttttggcaagaattattatcttcagacagctttaagtctctggctcctagccttgatgcaccctggaacaagggatcaaggggcctgaagactgtgaaacctctggcatcaccggcgttgaatggccccgctgatatcgcatcccttcccggcttccaagacaccttcacttccagcttcagcttcatccaactctcccttggtgctgctggagaacgcggagaagcagaaggttgcctgccatccagagaggccgaacctctgcatcagaggccccaagagatggcagctgaagcatctagctcagacaggccccacggggatcctcggcatctctggaccttcagtcttcacgctgctccaggcttggcggacttggctcaggtgacaaggagcagcagcaggcaatcagaatgtggcacggtctcctcctcctcctcggacactggcttctcttcccaggatgcatcctccgctggtgggcggggcgaccagggcggcggctgggccgatgcccatggatggcatacattgctcagggaatgggagcccatgctgcaggactacctactgagcaaccgcaggcagctggaggtcacttccttaattttaaagcttcagaaatgtcaagaaaaagtggtcgaggatggcgattacgatactgcagagacattgagacagaggttggaagaactggaacaggagaaaggccgcctgtcctgggctctgccttcacagcaacctgctcttcgcagcttcttgggttacctggcagcacagatacaggtggccttgcatggagccacccaaagggccggcagcgatgatccagaagccccacttgaaggacagctgaggactaccgcccaggatagcctgcctgcatccatcaccaggagggactggcttattcgagagaaacagcgattgcagaaggaaatcgaagctctccaagcacggatgtctgcgctggaggcaaaggaaaaacggctgagccaagagttggaggagcaggaggtgctgctccggtggccgggctgtgacctgatggcactggtggcccagatgtccccaggccagctgcaggaggtcagcaaggccttgggagagaccctgacctctgccaaccaggctcccttccaggtggagccacctgagaccctcaggagcctccgggaaaggacaaaatcactgaacctggctgtcagagaactcactgctcaggtgtgctcaggtgagaagctgtgcagctctctgaggaggagactcagtgacctcgacaccaggctgcctgccttgctggaagccaagatgctggccctatcaggaagctgcttctccacagccaaggagctcacggaggagatttgggccttgtcgtcagagcgggaagggctagagatgttcctgggcaggctgttggcactcagctccaggaacagcagaaggctgggcatcgtcaaagaggatcacctcaggtgcaggcaggacctggcactccaggacgccgcccacaaaacacgcatgaaggcaaacactgtgaagtgcatggaagtgttggaaggtcagctgagcagctgcaggtgcccgctgcttgggagagtgtggaaagcagacttggagacttgtcagttgctaatgcagagcctgcagcttcaggaagcaggcagcagcccacacgcagaggacgaggagcaggtgcatagcacaggagaggccgcccagacagctgctctggctgtccctcgaacaccccaccccgaagaagaaaagtcccccttgcaggtgctccaggagtgggacacccactcagctctttcaccacactgtgctgcaggcccatggaaagaggattctcacatcgtttctgctgaagttggagaaaagtgcgaagccataggcgtgaagctcctacacctggaagaccagcttctcggagccatgtacagtcacgatgaggctctctttcagtctctccagggggagctccagacggtgaaggaaacactgcaggccatgatcctgcagctccagccaacaaaggaggcaggagaggcctcagcttcctatccgacagctggtgctcaggaaaccgaggcctgaggt

(19) United States
(12) Patent Application Publication
(10) Pub. No.: US 2019/0167761 A1
(43) Pub. Date: Jun. 6, 2019

(54) PHARMACEUTICAL COMPOSITION FOR PREVENTING OR TREATING STRESS-RELATED DISEASE, INCLUDING DISC1 PROTEIN OR GENE ENCODING THE SAME

(71) Applicant: POSTECH ACADEMY-INDUSTRY FOUNDATION, Pohang-si (KR)

(72) Inventors: Sang Ki PARK, Pohang-si (KR); Nhung TRUONG, Pohang-si (KR); Sung Jin PARK, Pohang-si (KR); Yeong Jun SUH, Gwangju (KR); Cana PARK, Daegu (KR)

(21) Appl. No.: 16/197,491

(22) Filed: Nov. 21, 2018

(30) Foreign Application Priority Data
Dec. 1, 2017 (KR) 10-2017-0164209
May 29, 2018 (KR) 10-2018-0061085

(51) Int. Cl. A61K 38/17; A61P 25/24; A61P 25/22; G01N 33/50; A61K 35/30; C12Q 1/02

(52) U.S. Cl. CPC A61K 38/1709; A61P 25/24; A61P 25/22; A61K 48/005; A61K 35/30; C12Q 1/025; G01N 33/5008

(57) ABSTRACT

Provided herein are a pharmaceutical composition for preventing or treating a stress-related disease, which includes the DISC1 protein or a gene encoding the DISC protein, and a method of screening a material for preventing or treating the stress-related disease. As a result of studying the association between DISC1 and psychological stress, the inventors of the present disclosure verified the function of DISC1 in downregulating ER-mitochondria Ca²⁺ transfer induced by stress hormone-mediated oxidative stress by competitively inhibiting the binding of IP₃ to inositol 1,4,5-trisphosphate (IP₃) receptor type1 (IP₃R1) by binding to the IP₃R1 at the MAM, and an acting site of DISC1, and this provides a model of intracellular calcium response to physiological stress, and DISC1, a stress modulating substance, and the model may be usefully used in related fields for the prevention and treatment of stress-related diseases.

### CROSS-REFERENCE TO RELATED APPLICATION

This application claims priority to and the benefit of Korean Patent Application No. 2017-0164209, filed on Dec. 1, 2017 and Korean Patent Application No. 2018-0061085, filed on May 1, 2018, the disclosure of which are incorporated herein by reference in its entirety.

### BACKGROUND

### 1. Field of the Invention

The present disclosure relates to a pharmaceutical composition for preventing or treating a stress-related disease, which includes the disrupted in schizophrenia 1 (DISC1) protein or a gene encoding the DISC1 protein, and a method of screening a material for preventing or treating a stress-related disease.

### 2. Discussion of Related Art

The mitochondria-associated endoplasmic reticulum membrane (MAM) is a specialized sub-compartment that causes the endoplasmic reticulum (ER) and mitochondria to be in close proximity. Through electron tomographic analysis, it has been known that a very small distance (10 nm to 25 nm) exists between the MAM and a mitochondrial membrane, and many chaperones and several major Ca²⁺ channels, which are involved in intracellular Ca²⁺ homeostasis, are concentrated in the MAM. In addition, inositol-1,4,5-trisphosphate receptors (IP₃Rs) and voltage-dependent anion channels (VDACs) are abundant in the MAM and are physically bound by glucose-regulated protein 75, and consequently, Ca²⁺ stored in the ER is rapidly and efficiently transferred into mitochondria through the MAM.

Neurons are highly polarized to best fit the function for cell-to-cell communication. The ER and mitochondria are extensively distributed throughout the cell body and distal parts of neurites, and function as key components of neuronal Ca²⁺ signaling. ER Ca²⁺ channels regulate various neuron-specific processes, such as synaptic plasticity and neurotransmitter release, and the ER and mitochondria are also very closely associated with the postsynaptic density (PSD), which supplies ATP in a Ca²⁺-responsive manner, in that mitochondrial ATP production appears to be tightly regulated by Ca²⁺ levels, and this suggests the potential importance of the MAM in neurons. Indeed, several pieces of evidence suggest that ER-mitochondria connection via MAM and many related functions are abnormal in neurological diseases such as Alzheimer's disease and amyotrophic lateral sclerosis, which display some common features, such as mitochondrial dysfunction and intracellular Ca²⁺ homeostasis collapse.

Oxidative stress induces ER-mitochondria Ca²⁺ transfer at the MAM. Hydrogen peroxide (H₂O₂), the superoxide anion (O⁻²), and C2-ceramide, which cause oxidative stress, trigger Ca²⁺ release from the ER via IP₃R, leading to its transfer into mitochondria. Oxidative stress-induced mitochondrial Ca²⁺ accumulation reportedly contributes to mitochondrial depolarization and changes in oxidative phosphorylation, and this is reported to be blocked by the addition of ER Ca²⁺ channel blockers. Oxidative stress is drawing attention because it is a key mechanism that underlies various psychological stress-induced cellular and intracellular responses. In addition, it is reported that short- and long-term treatment with cortisol and other glucocorticoids, which are physiological stress hormones released in response to psychological stress, result in the impairment of oxidative energy metabolism and inhibition of antioxidation pathways, causing mitochondrial energy deficits and a drastic increase in cellular reactive oxygen species (ROS), and eventually induces oxidative stress in the brain.

Meanwhile, disrupted in schizophrenia 1 (DISC1) has been studied in the analysis of various major mental illnesses, including schizophrenia, in association with a chromosomal translocation by which the open reading frame for DISC1 is affected. Subsequent studies have provided evidence that functional collapse of the DISC1 protein underlies the pathology of a wide range of major metal illnesses beyond the individual disease category. For example, DISC1 mutant animal models display a variety of behavioral phenotypes, including deficits in cognitive memory and social behavioral deficits, which are relevant to endophenotypes of major psychiatric disorders (Neuron 54, 387-402).

Therefore, the inventors of the present disclosure assumed that DISC1 could participate in the interaction between environmental risk factors such as psychological stress and intracellular calcium cascades, and to verify this hypothesis, they investigated MAM localization of DISC1 and its influence on Ca²⁺ transfer between the ER and mitochondria under physiologically- and pathologically-related conditions.

### SUMMARY OF THE INVENTION

Under this assumption, as a result of studying the association between DISC1 and response in neurons according to psychological stress, it was verified that DISC1 competitively inhibited the binding of IP₃ to inositol 1,4,5-trisphosphate (IP₃) receptor type 1 (IP₃R1) by binding to the IP₃R1 at the MAM, thereby downregulating endoplasmic reticulum-mitochondria calcium ion (Ca²⁺) transfer induced by stress hormone-mediated oxidative stress, thus completing the present invention based on this finding.

Therefore, one embodiment of the present disclosure provides a pharmaceutical composition for preventing or treating a stress-related disease, which includes, as an active ingredient, the disrupted in schizophrenia 1 (DISC1) protein or a gene encoding the DISC1 protein.

Another embodiment of the present disclosure provides a method of screening a material for preventing or treating a stress-related disease.

Additional aspects will be set forth in part in the description which follows and, in part, will be apparent from the description, or may be learned by practice of the presented embodiments.

According to an aspect of one embodiment, a pharmaceutical composition for preventing or treating a stress-related disease includes the disrupted in schizophrenia 1 (DISC1) protein or a gene encoding the DISC1 protein as an active ingredient.

In one embodiment, the stress-related disease may be any one selected from the group consisting of sleep disorders, depression, adaptive disorders, eating disorders, and anxiety disorders.

In another embodiment, the gene may be inserted into a plasmid expression vector or a viral vector.

In another embodiment, the DISC1 protein may regulate endoplasmic reticulum-mitochondria Ca²⁺ transfer induced by stress hormone-mediated oxidative stress at the mitochondria-associated endoplasmic reticulum membrane (MAM).

In another embodiment, the DISC1 protein may regulate Ca²⁺ transfer by competitively inhibiting the binding of IP₃ to inositol 1,4,5-trisphosphate (IP₃) receptor type1 (IP₃R1) by binding to the IP₃R1 at the MAM.

In another embodiment, the stress hormone may be a glucocorticoid.

According to an aspect of another embodiment, a method of screening a material for preventing or treating a stress-related disease includes:

(a) treating cells expressing a DISC1 protein or a gene encoding the DISC1 protein with a candidate material in vitro;

(b) measuring an expression level or activity of the DISC1 protein in the cells; and

(c) selecting, as a material for preventing or treating a stress-related disease, a material that increases the expression level or activity of the DISC1 protein as compared to a group that is not treated with the candidate material.

In one embodiment, the cells may be neurons.

In another embodiment, the candidate material may be selected from the group consisting of a compound, a microorganism culture or extract, a natural extract, a nucleic acid, and a peptide.

In another embodiment, the nucleic acid may be selected from the group consisting of siRNA, shRNA, microRNA, antisense RNA, an aptamer, a locked nucleic acid (LNA), a peptide nucleic acid (PNA), and a morpholino.

In another embodiment, in the measuring process, the expression level may be measured using one or more methods selected from the group consisting of western blotting, radioimmunoassay (RIA), radioimmunodiffusion, enzyme-linked immunosorbent assay (ELISA), immunoprecipitation, flow cytometry, immunofluorescence, Ouchterlony double immunodiffusion, a complement fixation assay, and a protein chip.

In another embodiment, (b) in the measuring process, the activity may be measured by measuring a degree to which the DISC1 protein decreases endoplasmic reticulum-mitochondria Ca²⁺ transfer by competitively inhibiting the binding of IP₃ to IP₃R1 by binding to the IP₃R1 at the MAM.

According to an aspect of another embodiment, a method of preventing or treating a stress-related disease includes administering the pharmaceutical composition to a subject.

According to an aspect of another embodiment, there is provided a use of the pharmaceutical composition for preventing or treating a stress-related disease.

### BRIEF DESCRIPTION OF THE DRAWINGS

These and/or other aspects will become apparent and more readily appreciated from the following description of the embodiments, taken in conjunction with the accompanying drawings in which:

FIG. 1A illustrates results showing the MAM localization of the DISC1 protein specifically, illustrates immunoblotting results identifying the presence of DISC1 expression in cell organelle fractions of cerebral cortical neurons (MAM+Mito (DISC1 LI), MAM+Mito, MAM, ER, Mito, Whole lysate);

FIG. 1B illustrates results showing the MAM localization of the DISC1 protein specifically, illustrates results of immunofluorescence analysis for identifying the positions of DISC1, ER, and mitochondria;

FIG. 1C illustrates results showing the MAM localization of the DISC1 protein specifically, illustrates immunofluorescence analysis and immunoblotting results showing the importance of residues 1-201 of DISC1 in MAM localization; and

FIG. 1D illustrates results showing the MAM localization of the DISC1 protein specifically, illustrates immunofluorescence analysis and immunoblotting results showing the importance of residues 1-201 of DISC1 in MAM localization;

FIG. 2A illustrates identification results showing the effect of IP₃R1 on DISC1 located in the MAM specifically, illustrates results showing the interaction between IP₃R1 and DISC1 at the MAM;

FIG. 2B illustrates identification results showing the effect of IP₃R1 on DISC1 located in the MAM specifically, illustrates results showing the interaction between IP₃R1 and DISC1 at the MAM;

FIG. 2C illustrates identification results showing the effect of IP₃R1 on DISC1 located in the MAM specifically, illustrates results showing the interaction between IP₃R1 and DISC1 at the MAM;

FIG. 2D illustrates identification results showing the effect of IP₃R1 on DISC1 located in the MAM specifically, illustrates results showing a change in DISC1 expression according to an expression level of IP₃R1 at the MAM; and

FIG. 2E illustrates identification results showing the effect of IP₃R1 on DISC1 located in the MAM specifically, illustrates results showing a change in DISC1 expression according to an expression level of IP₃R1 at the MAM;

FIG. 3A illustrates verification results showing that DISC1 inhibited binding between IP₃R1 and an IP₃R1 ligand specifically, illustrates immunoprecipitation analysis results showing the interaction between each functional domain of IP₃R1 and the DISC1 protein; and

FIG. 3B illustrates verification results showing that DISC1 inhibited binding between IP₃R1 and an IP₃R1 ligand specifically, illustrates competitive IP₃ binding analysis results showing an effect of DISC1 on binding between IP₃R1 and IP₃;

FIG. 4A illustrates verification results showing that DISC1 present in the MAM was involved in ER-mitochondria Ca²⁺ transfer specifically, illustrates results showing that mitochondrial Ca²⁺ accumulation was sharply increased when DISC1 expression was inhibited in a case in which IP₃ was exposed to permeable neurons; and

FIG. 4B illustrates verification results showing that DISC1 present in the MAM was involved in ER-mitochondria Ca²⁺ transfer specifically, illustrates results showing a decrease in mitochondrial Ca²⁺ accumulation increase upon DISC1 overexpression or the expression of DISC1 bound to an ER membrane protein (UBC6-DISC1);

FIG. 5A illustrates verification results showing that ER-mitochondria Ca²⁺ transfer regulated by DISC1 mainly occurred at the MAM Specifically, illustrates results showing that mitochondrial Ca²⁺ accumulation was decreased to a level of a control by simultaneously inhibiting MFN2 expression in neurons in which DISC1 expression was inhibited;

FIG. 5B illustrates verification results showing that ER-mitochondria Ca²⁺ transfer regulated by DISC1 mainly occurred at the MAM Specifically, illustrates results showing a significant increase in mitochondrial Ca²⁺ accumulation upon inducing the enhancement of ER-mitochondria contact by a rapamycin-inducible bridge-forming module in neurons in which DISC1 was overexpressed;

FIG. 5C illustrates verification results showing that ER-mitochondria Ca²⁺ transfer regulated by DISC1 mainly occurred at the MAM Specifically, illustrates results showing changes in ER-mitochondria Ca²⁺ transfer according to a change in expression level of DISC1 upon treatment with IP₃ in neuroblastoma CAD cell-derived crude MAM fractions; and

FIG. 5D illustrates verification results showing that ER-mitochondria Ca²⁺ transfer regulated by DISC1 mainly occurred at the MAM Specifically, illustrates results showing changes in ER-mitochondria Ca²⁺ transfer according to a change in expression level of DISC1 upon treatment with IP₃ in neuroblastoma CAD cell-derived crude MAM fractions;

FIG. 6A illustrates verification results showing that DISC1 regulated oxidative stress-mediated ER-mitochondria Ca²⁺ transfer specifically, illustrates measurement results of mitochondrial Ca²⁺ levels of a control (CTL shRNA) and neurons in which DISC1 expression was inhibited (DISC1 shRNA) upon treatment with hydrogen peroxide (H₂O₂) or MSC, which is a glutathione peroxidase inhibitor;

FIG. 6B illustrates verification results showing that DISC1 regulated oxidative stress-mediated ER-mitochondria Ca²⁺ transfer specifically, illustrates measurement results of mitochondrial Ca²⁺ levels of the neurons of the respective groups upon treatment with 2-APB, which is an IP₃R inhibitor, under the same conditions as described above;

FIG. 6C illustrates verification results showing that DISC1 regulated oxidative stress-mediated ER-mitochondria Ca²⁺ transfer specifically, illustrates measurement results of mitochondrial Ca²⁺ levels of neurons in which DISC1 or mutant DISC1 was overexpressed, upon treatment with H₂O₂;

FIG. 6D illustrates verification results showing that DISC1 regulated oxidative stress-mediated ER-mitochondria Ca²⁺ transfer specifically, illustrates measurement results of mitochondrial Ca²⁺ levels according to H₂O₂ treatment upon inducing the enhancement of ER-mitochondria contact by a rapamycin-inducible bridge-forming module in neurons in which DISC1 was overexpressed; and

FIG. 6E illustrates verification results showing that DISC1 regulated oxidative stress-mediated ER-mitochondria Ca²⁺ transfer specifically, illustrates measurement results of mitochondrial Ca²⁺ levels by H₂O₂ treatment in embryo-derived cerebral cortical neurons having an impaired DISC1 locus (DISC1 LI);

FIG. 7A illustrates verification results showing that DISC1 was closely related to dysfunction induced by oxidative stress-mediated mitochondrial Ca²⁺ accumulation specifically, illustrates changes in mitochondrial membrane potential by treatment of H₂O₂ in a time-dependent manner or a concentration-dependent manner when DISC1 expression was inhibited;

FIG. 7B illustrates verification results showing that DISC1 was closely related to dysfunction induced by oxidative stress-mediated mitochondrial Ca²⁺ accumulation specifically, illustrates changes in mitochondrial membrane potential by treatment of H₂O₂ in a time-dependent manner or a concentration-dependent manner when DISC1 expression was inhibited;

FIG. 7C illustrates verification results showing that DISC1 was closely related to dysfunction induced by oxidative stress-mediated mitochondrial Ca²⁺ accumulation specifically, illustrates measurement results of the degree of ROS generation;

FIG. 7D illustrates verification results showing that DISC1 was closely related to dysfunction induced by oxidative stress-mediated mitochondrial Ca²⁺ accumulation specifically, illustrates measurement results of the degree of ROS generation;

FIG. 7E illustrates verification results showing that DISC1 was closely related to dysfunction induced by oxidative stress-mediated mitochondrial Ca²⁺ accumulation specifically, illustrates measurement results of the degree of ROS generation upon treatment of neurons in which DISC1, which is expressed in the ER-MAM, was overexpressed or mutant DISC1 (DISC1 Δ 1-201) was expressed, with H₂O₂;

FIG. 7F illustrates verification results showing that DISC1 was closely related to dysfunction induced by oxidative stress-mediated mitochondrial Ca²⁺ accumulation specifically, respectively illustrates mitochondrial membrane potential and the degree of ROS generation by treatment with H₂O₂ in embryo-derived cerebral cortical neurons having an impaired DISC1 locus (DISC1 LI); and

FIG. 7G illustrates verification results showing that DISC1 was closely related to dysfunction induced by oxidative stress-mediated mitochondrial Ca²⁺ accumulation specifically, respectively illustrates mitochondrial membrane potential and the degree of ROS generation by treatment with H₂O₂ in embryo-derived cerebral cortical neurons having an impaired DISC1 locus (DISC1 LI);

FIG. 8A illustrates verification results showing increases in mitochondrial Ca²⁺ transfer in the ER dependent on corticosterone-mediated oxidative stress specifically, illustrates measurement results of the degree of ROS generation by treatment of cerebral cortical neurons with corticosterone;

FIG. 8B illustrates verification results showing increases in mitochondrial Ca²⁺ transfer in the ER dependent on corticosterone-mediated oxidative stress specifically, illustrates measurement results of mitochondrial Ca²⁺ levels by treatment with APO, which is an antioxidant, and treatment with 2-APB, which is an IP₃R inhibitor, respectively under the same conditions; and

FIG. 8C illustrates verification results showing increases in mitochondrial Ca²⁺ transfer in the ER dependent on corticosterone-mediated oxidative stress specifically, illustrates measurement results of mitochondrial Ca²⁺ levels by treatment with APO, which is an antioxidant, and treatment with 2-APB, which is an IP₃R inhibitor, respectively under the same conditions;

FIG. 9A illustrates verification results showing that DISC1 downregulated increases in ER-mitochondria Ca²⁺ transfer in a corticosterone-dependent manner specifically, illustrates measurement results of mitochondrial Ca²⁺ levels according to a change in an expression level of DISC1 by treatment of neurons with corticosterone (CORT);

FIG. 9B illustrates verification results showing that DISC1 downregulated increases in ER-mitochondria Ca²⁺ transfer in a corticosterone-dependent manner specifically, illustrates measurement results of mitochondrial Ca²⁺ levels according to a change in an expression level of DISC1 by treatment of neurons with corticosterone (CORT);

FIG. 9C illustrates verification results showing that DISC1 downregulated increases in ER-mitochondria Ca²⁺ transfer in a corticosterone-dependent manner specifically, illustrates measurement results of mitochondrial Ca²⁺ levels according to a change in an expression level of DISC1 by treatment of neurons with corticosterone (CORT);

FIG. 9D illustrates verification results showing that DISC1 downregulated increases in ER-mitochondria Ca²⁺ transfer in a corticosterone-dependent manner specifically, illustrates measurement results of the degree of ROS generation;

FIG. 9E illustrates verification results showing that DISC1 downregulated increases in ER-mitochondria Ca²⁺ transfer in a corticosterone-dependent manner specifically, illustrates measurement results of the degree of ROS generation;

FIG. 9F illustrates verification results showing that DISC1 downregulated increases in ER-mitochondria Ca²⁺ transfer in a corticosterone-dependent manner specifically, illustrates measurement results of the degree of ROS generation;

FIG. 9G illustrates verification results showing that DISC1 downregulated increases in ER-mitochondria Ca²⁺ transfer in a corticosterone-dependent manner specifically, illustrates measurement results of the degree of ROS generation by treatment with corticosterone in embryo-derived cerebral cortical neurons having an impaired DISC1 locus (DISC1 LI);

FIG. 9H illustrates verification results showing that DISC1 downregulated increases in ER-mitochondria Ca²⁺ transfer in a corticosterone-dependent manner specifically, illustrates measurement results of the degree of ROS generation by treatment with corticosterone in embryo-derived cerebral cortical neurons having an impaired DISC1 locus (DISC1 LI); and

FIG. 10 depicts the function of DISC1, which was verified in the present disclosure, in an ER-mitochondria Ca²⁺ transfer mechanism induced by stress hormone-mediated oxidative stress.

### DETAILED DESCRIPTION OF EXEMPLARY EMBODIMENTS

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

The inventors of the present disclosure verified a novel function of DISC1 in a calcium transfer mechanism mediated by oxidative stress due to the secretion of a stress hormone, thus completing the present disclosure based on this finding.

Therefore, the present disclosure provides a pharmaceutical composition for the prevention or treatment of a stress-related disease, which includes the disrupted in schizophrenia 1 (DISC1) protein or a gene encoding the DISC protein as an active ingredient.

The term “stress” as used herein refers to a non-specific biological response of the body against multiple injuries and stimuli in the body, and was first coined by Hans Selye, who is a Canadian endocrinologist. The stress response is intended to protect the body by the secretion of adrenaline, which is a stimulating hormone, or other stress hormones into the blood, and is accompanied by physical symptoms such as fatigue, headaches, insomnia, and muscle aches; mental symptoms such as concentration or memory loss, emptiness, and confusion; emotional symptoms such as anxiety, depression, nervousness, and frustration; and behavioral symptoms such as restlessness and nervous behavior. Excessive or long-lasting stress may lead to stress-induced mental illness.

In the present disclosure, the stress-related disease may include all diseases that may be caused by stress, more preferably a disease caused by a physical effect due to the secretion of a stress hormone, and more preferably any one selected from the group consisting of sleep disorders, depression, adaptive disorders, eating disorders, and anxiety disorders, but the present disclosure is not limited thereto.

In the present disclosure, the stress hormone may be a glucocorticoid, more preferably cortisone, cortisol, corticosterone, or the like, but the present disclosure is not limited thereto.

The term “prevention” as used herein means all actions that inhibit stress-related diseases or delay the onset thereof via administration of the pharmaceutical composition according to the present disclosure.

The term “treatment” as used herein means all actions that alleviate or beneficially change symptoms due to stress-related diseases via administration of the pharmaceutical composition according to the present disclosure.

The DISC1 protein and the gene encoding the protein, according to the present disclosure, may be one or more selected from amino acid sequence information and base sequence information of human-derived DISC1, shown in Table 1 below, or may be derived from a mouse, and more preferably, the DISC1 protein may consist of an amino acid sequence of SEQ ID NO: 1 (NCBI accession number: NP_061132.2) or SEQ ID NO: 2 (NP_777279.2), and the gene encoding the DISC1 protein may consist of a base sequence of SEQ ID NO: 3 (NM_018662.2) or SEQ ID NO: 4 (NM_174854.2), but the present disclosure is not limited thereto.

TABLE 1

| DISC1 isoform | mRNA | Protein |
|---|---|---|
| DISC1 isoform L | NM_018662.2 | NP_061132.2 |
| DISC1 isoform Lv | NM_001012957.1 | NP_001012975.1 |
| DISC1 isoform Es | NM_001012958.1 | NP_001012976.1 |
| DISC1 isoform S | NM_001012959.1 | NP_001012977.1 |
| DISC1 isoform a | NM_001164537.1 | NP_001158009.1 |
| DISC1 isoform b | NM_001164538.1 | NP_001158010.1 |
| DISC1 isoform c | NM_001164539.1 | NP_001158011.1 |
| DISC1 isoform d | NM_001164540.1 | NP_001158012.1 |
| DISC1 isoform e | NM_001164541.1 | NP_001158013.1 |
| DISC1 isoform f | NM_001164542.1 | NP_001158014.1 |
| DISC1 isoform g | NM_001164544.1 | NP_001158016.1 |
| DISC1 isoform h | NM_001164545.1 | NP_001158017.1 |
| DISC1 isoform i | NM_001164546.1 | NP_001158018.1 |
| DISC1 isoform j | NM_001164547.1 | NP_001158019.1 |
| DISC1 isoform k | NM_001164548.1 | NP_001158020.1 |
| DISC1 isoform l | NM_001164549.1 | NP_001158021.1 |
| DISC1 isoform m | NM_001164550.1 | NP_001158022.1 |
| DISC1 isoform n | NM_001164551.1 | NP_001158023.1 |
| DISC1 isoform o | NM_001164552.1 | NP_001158024.1 |
| DISC1 isoform p | NM_001164553.1 | NP_001158025.1 |
| DISC1 isoform q | NM_001164554.1 | NP_001158026.1 |
| DISC1 isoform r | NM_001164555.1 | NP_001158027.1 |
| DISC1 isoform t | NM_001164556.1 | NP_001158028.1 |
| DISC1 isoform L | NM_018662.2 | NP_061132.2 |

The gene may be inserted into a plasmid expression vector or a viral vector, but the present disclosure is not limited thereto.

The inventors of the present disclosure verified a novel function of DISC1, which is associated with stress, through examples.

In one embodiment, as a result of analyzing whether or not DISC1 was expressed in cell organelles obtained such that brains were extracted from adult mice and sequential fractionation was performed thereon, it was confirmed that DISC1 was located at the MAM and this was governed by residues 1-201 of DISC1 (see Example 2).

In another embodiment, as a result of examining the correlation between IP₃R1 and DISC1, which are known to be abundant in the MAM, it was confirmed that DISC1 levels in the MAM varied depending on an expression level of IP₃R1, and DISC1 bound to ligand-binding and modulatory domains of IP₃R1, thereby competitively inhibiting the binding of IP₃ thereto (see Example 3).

In another embodiment, as a result of analyzing whether or not DISC1 and IP₃R1 were involved in endoplasmic reticulum (ER)-mitochondria Ca²⁺ transfer at the MAM by consideration of the functional association therebetween, it was confirmed that mitochondrial Ca²⁺ levels varied depending on an expression level of DISC1 in cells upon enhancing the cell permeability of neurons and being exposed to IP₃, and DISC1 regulated Ca²⁺ transfer from the ER via IP₃R1. In addition, it was confirmed through additional experiments that DISC1 regulated Ca²⁺ transfer from the MAM (see Example 4).

In another embodiment, since oxidative stress has been reported to induce Ca²⁺ release from the ER and mitochondrial Ca²⁺ transfer in various cells including neurons, it was analyzed whether or not DISC1 was involved in these processes. As a result of analysis, it was confirmed that, when neurons were exposed to H₂O₂, ER-mitochondria Ca²⁺ transfer varied depending on an expression level or variation of DISC1 in the cells (see Example 5).

In another embodiment, since the excessive accumulation of Ca²⁺ in mitochondria due to oxidative stress may cause mitochondrial dysfunction, it was analyzed whether or not DISC1 affected this process. As a result of analysis, it was confirmed that, when neurons were exposed to H₂O₂, mitochondrial membrane potential and the degree of reactive oxygen species (ROS) generation varied depending on an expression level or variation of DISC1 in the cells, from which it was confirmed that the function of DISC1 at the MAM was closely associated with oxidative stress-induced mitochondrial function (see Example 6).

In another embodiment, as a result of examining the correlation between DISC1 and glucocorticoids based on existing findings showing the induction of oxidative stress in neurons by stimulation of an excess amount of a glucocorticoid, which is a stress hormone, it was observed that, when neurons were treated with corticosterone, which is one of the glucocorticoids, the levels of ROS and mitochondrial Ca²⁺ were increased in proportion to a treatment concentration (see Example 7). In addition, as a result of examining the correlation between the phenomenon and DISC1, it was confirmed that the levels of ROS and mitochondrial Ca²⁺ varied depending on an expression level or variation of DISC1 (see Example 8).

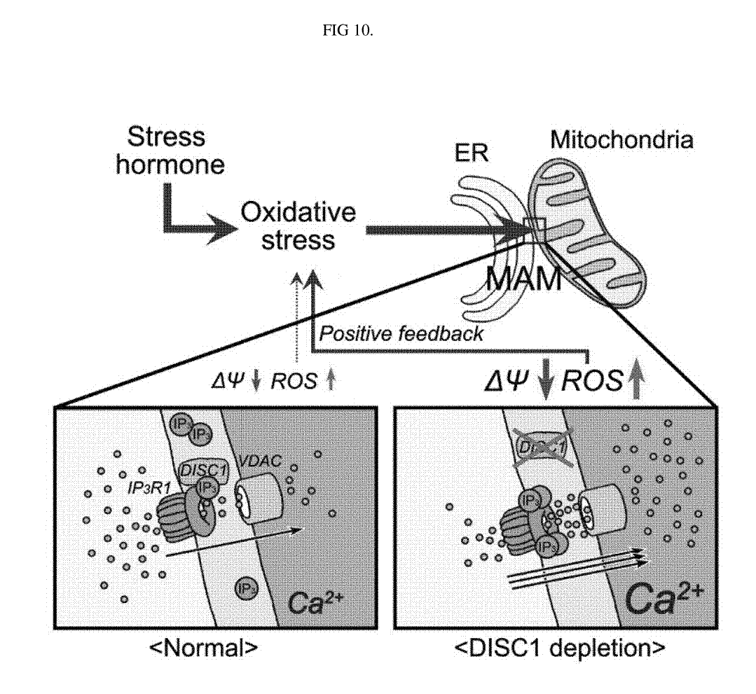

Taken together, the above results indicate that, as illustrated in FIG. 10, Ca²⁺ transfer through the MAM may be increased by oxidative stress according to stress hormone secretion, and thus mitochondria dysfunction may be caused, but in this process, DISC1 downregulates the Ca²⁺ transfer by binding to IP₃R1 on the side of the ER of the MAM, and this suggests that the DISC1 protein or the gene encoding the DISC1 protein may be usefully used for the prevention or treatment of a stress-related disease.

The pharmaceutical composition according to the present disclosure includes the DISC1 protein or a gene encoding the DISC1 protein as an active ingredient, and may further include a pharmaceutically acceptable carrier. The pharmaceutically acceptable carrier includes saline, sterilized water, Ringer's solution, buffered saline, cyclodextrin, a dextrose solution, a maltodextrin solution, glycerol, ethanol, liposomes, and the like, which are commonly used in formulation, but the present disclosure is not limited thereto, and may further include, if needed, other general additives such as an antioxidant, a buffer solution, and the like. In addition, the pharmaceutical composition may be formulated into an injectable preparation such as an aqueous solution, a suspension, an emulsion, or the like, pills, capsules, granules, or tablets by additionally adding a diluent, a dispersant, a surfactant, a binder, a lubricant, or the like. Suitable pharmaceutically acceptable carriers and formulations are described in detail in Remington's document and these carriers may be formulated according to each ingredient using a method disclosed in the document. Dosage forms of the pharmaceutical composition of the present disclosure are not particularly limited, but may include an injection, an inhalant, a composition for external application to the skin, or the like.

The pharmaceutical composition of the present disclosure may be administered orally or parenterally (e.g., administered intravenously, subcutaneously, intraperitoneally, or locally) according to the purpose of use, but preferably, may be administered orally, and a suitable dose of the pharmaceutical composition may vary depending on the condition and body weight of patients, the severity of disease, dosage forms, administration routes, and administration time, but may be appropriately selected by those of ordinary skill in the art.

The pharmaceutical composition of the present disclosure is administered in a pharmaceutically effective amount. The term “pharmaceutically effective amount” as used herein refers to an amount sufficient to treat or diagnose diseases at a reasonable benefit/risk ratio applicable to medical treatment or diagnosis, and an effective dosage level may be determined according to factors including type of diseases of patients, the severity of disease, the activity of drugs, sensitivity to drugs, administration time, administration routes, excretion rate, treatment periods, and simultaneously used drugs, and other factors well known in the medical field. The pharmaceutical composition according to the present disclosure may be administered as an individual therapeutic agent or may be administered in combination with other therapeutic agents, may be administered sequentially or simultaneously with existing therapeutic agents, and may be administered in a single dose or multiple doses. Taking all the factors into consideration, it is important to administer the pharmaceutical composition in a minimum amount sufficient to obtain a maximum effect without side effects, and this may be easily determined by those of ordinary skill in the art.

In particular, the effective amount of the pharmaceutical composition of the present disclosure may vary depending on the age, gender, condition, and body weight of patients, the bioavailability of an active ingredient, inactivity, excretion rate, the type of diseases, simultaneously used drugs, and the pharmaceutical composition may generally be administered at a dose of about 0.001 mg to about 150 mg, preferably about 0.01 mg to about 100 mg, per kg of body weight, daily or every other day, or once to three times a day. However, the effective amount may be increased or decreased according to administration routes, the severity of obesity, gender, body weight, age, and the like, and thus the dosage is not intended to limit the scope of the present disclosure in any way.

According to another embodiment of the present disclosure, there is provided a method of screening a material for the prevention or treatment of a stress-related disease, including the following processes:

(a) treating cells expressing the disrupted in schizophrenia 1 (DISC1) protein or a gene encoding the DISC1 protein with a candidate material in vitro;

(b) measuring an expression level or activity of the DISC1 protein in the cells; and

(c) selecting, as a material for the prevention or treatment of a stress-related disease, a material that increases the expression level or activity of the DISC1 protein as compared to a group that is not treated with the candidate material.

In the present disclosure, the cells may be neurons, but the present disclosure is not limited thereto.

In the present disclosure, the candidate material may be selected from the group consisting of a compound, a microorganism culture or extract, a natural extract, a nucleic acid, and a peptide, and the nucleic acid may be selected from the group consisting of siRNA, shRNA, microRNA, antisense RNA, an aptamer, a locked nucleic acid (LNA), a peptide nucleic acid (PNA), and a morpholino, but the present disclosure is not limited thereto.

In process (b), the expression level may be measured using one or more methods selected from the group consisting of western blotting, radioimmunoassay (RIA), radioimmunodiffusion, enzyme-linked immunosorbent assay (ELISA), immunoprecipitation, flow cytometry, immunofluorescence, Ouchterlony double immunodiffusion, a complement fixation assay, and a protein chip, but the present disclosure is not limited thereto.

In process (b), the activity may be measured by measuring a degree to which the DISC1 protein decreases ER-mitochondria Ca²⁺ transfer by competitively inhibiting the binding of IP₃ to IP₃R1 by binding to the IP₃R1 at the MAM, and the measurement may be appropriately performed using a method known in the art without limitation.

According to another embodiment of the present disclosure, there is provided a method of preventing or treating a stress-related disease, which includes administering, to a subject, a pharmaceutical composition including the disrupted in schizophrenia 1 (DISC1) protein or a gene encoding the DISC1 protein as an active ingredient.

According to another embodiment of the present disclosure, there is provided a use of the pharmaceutical composition for the prevention or treatment of a stress-related disease.

Hereinafter, exemplary examples will be described to aid in understanding of the present disclosure. However, these examples are only provided to more easily understand the present disclosure and are not intended to limit the scope of the present disclosure.

### EXAMPLES

### Example 1. Experimental Preparation and Experimental Methods

1-1. Experimental Animals

In an example of the present disclosure, pregnant C57BL/6 mice were purchased from Hyochang Science, and then cerebral cortical neurons were collected and cultured at the stage of embryonic day E15-16. Meanwhile, male WT mice (C57BL/6) and DISC1 locus-impaired mice (DISC1 LI, C57BL/6 strain) were fed arbitrarily and kept on a 12-hr light/12-hr dark cycle for 10 to 12 weeks, and brain lysates of the mice were used in experiments. All animal experiments of the present example were approved by the Committee for Laboratory Animal Care and Use of the Pohang University of Science and Technology, and were carried out in accordance with the approved guidelines.

1-2. Cellular Fractionation

Brains were isolated from three adult mice, homogenized, and centrifuged, and then a portion of the supernatant was stored as a whole lysate fraction, and the rest was further centrifuged at 13,800×g and 4° C. for 10 minutes. Subsequently, the pellet (crude MAM) was collected, and the supernatant was loaded on a sucrose gradient and centrifuged again, and the ER fraction, which appeared as a white band, was collected from the centrifuged product. The residual crude MAM pellet was loaded on a sucrose gradient and centrifuged, and a synaptosomal fraction, which appeared as the third white band, was collected, and the residual pellet was loaded on the top of a Percoll gradient and centrifuged, and then upper and lower bands were collected as an MAM fraction and a mitochondria fraction, respectively.

1-3. IP₃ Binding Assay

Transfected HEK293FT cells were lysed in a NP40 buffer, and then the proteins were immunoprecipitated with antibodies. The immunoprecipitated proteins were incubated with 3 nM [³H]-IP₃ at 4° C. for 1 hour and the concentration of cold IP₃ was increased in a Ca²⁺-free CLM buffer. Meanwhile, brains were isolated from adult WT or DISC1 LI mice and lysed in a NP40 buffer by sonication, and then endogenous IP₃R1 and the DISC1 protein were immunoprecipitated with specific antibodies, and then incubated with [³H]-IP₃ in the same manner as described above, and the concentration of cold IP₃ was increased. Thereafter, the resulting mixture was filtered with a GF/B filter and washed three times with a CLM buffer, and then the filter was dried, and radioactivity was measured using a scintillation counter.

1-4. Real-time Ca²⁺ Imaging Using GCaMP6s

Neurons (DIV7-8) transfected with Mito-GCaMP6 along with each construct were permeabilized with digitonin and Ca²⁺-free ionomycin at 37° C. for 2 minutes in a modified EGTA- and Ca²⁺-free buffer, and then was exposed to IP₃. To measure the effect of RiBFM on mitochondrial Ca²⁺ responses, neurons were transfected with Mito GCaMP6, AKAP1-FKBP12, and FRB-SAC1 together with pFlag-cmv2 or Flag-DISC1 on DIV5-6, and neurons pre-incubated with rapamycin for 5 minutes were exposed to IP₃ in the same manner as described above.

1-5. Statistical Analysis

Experimental data was subjected to one- or two-way ANOVA analysis by a Student's t test or a Bonferroni post-hoc test for the comparison between two different groups or the comparison between multiple groups, and then expressed as mean±SEM. Differences between the groups were considered to be significant when the p value is less than 0.05 (<0.05). No statistical methods were used to determine a sample size, and randomization was not used for analyses.

### Example 2. Identification of DISC1 Present at MAM

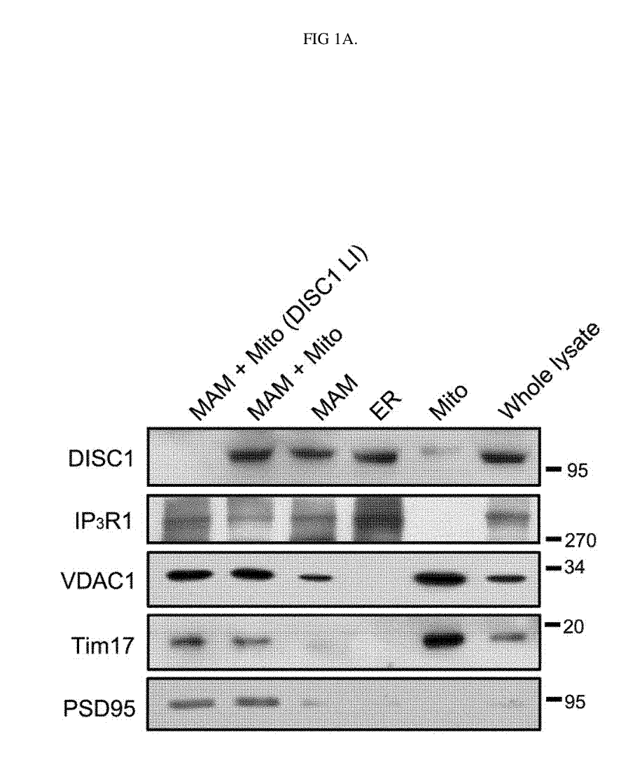

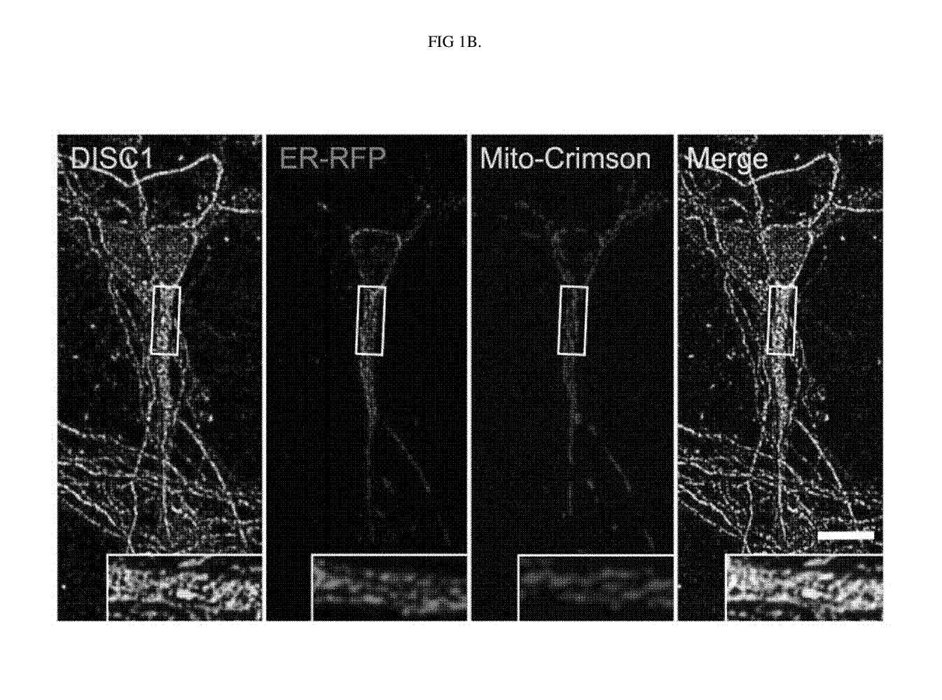

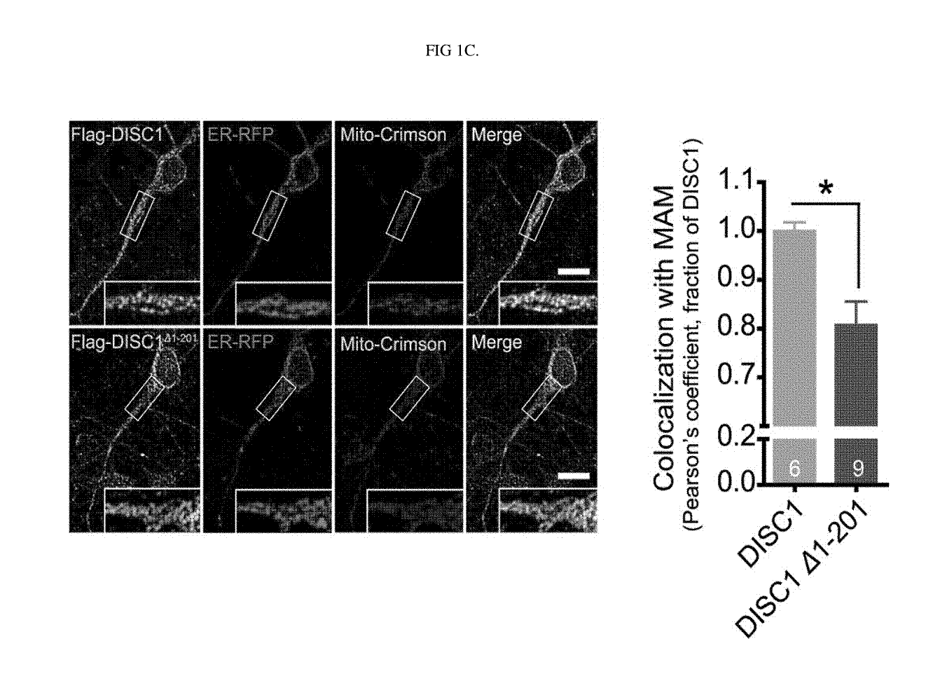

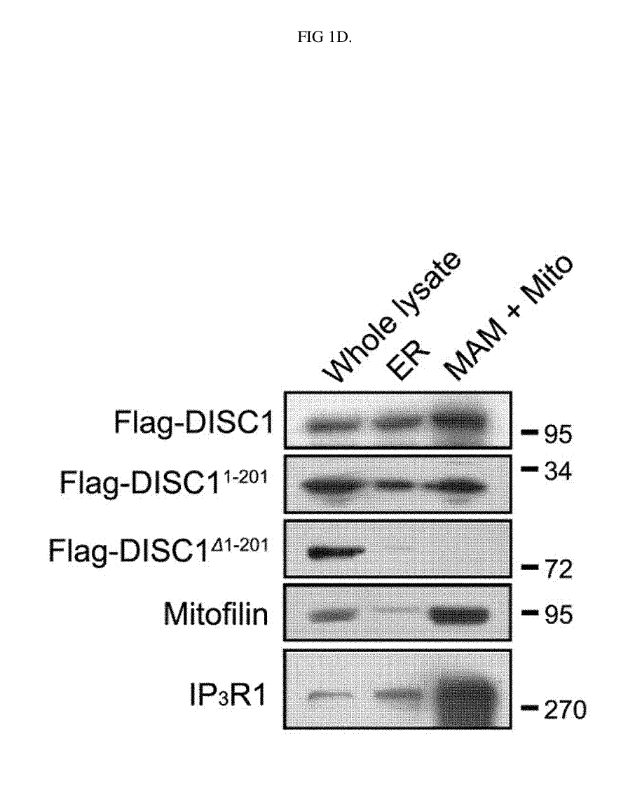

The inventors of the present disclosure examined the intracellular localization of DISC1 in brains of adult mice and mouse embryonic cerebral cortical neurons using biochemical and immunofluorescence techniques. For this, first, brains were isolated from adult mice, and serial subcellular fractionation was performed to isolate intracellular organelles. Subsequently, each fraction and the purity thereof were confirmed by immunoblotting, and expression levels of markers specific to organelles to be identified, i.e., IP3R1 (ER/MAM), VDAC1 (ER-associated mitochondria), Tim17 (mitochondria), and PSD95 (synaptosome) were measured. As a result, as illustrated in FIG. 1A, each fraction and the purity thereof were confirmed by the expression of the markers.

In addition, as a result of observing whether or not endogenous DISC1 was expressed in each fraction, as illustrated in FIG. 1A, the DISC1 protein was observed in a crude MAM fraction (MAM+Mito) in which mitochondria was attached to the MAM, and synaptosomes were observed in the crude MAM fraction, but not observed after further fractionation. At this time, the DISC1 protein was not observed in a crude MAM fraction derived from mice having an impaired DISC1 locus (MAM+Mito (DISC1 LI)), through which the specificity of DISC1 antibodies was confirmed. In addition, similarly to the previously reported results, it was confirmed that the endogenous DISC1 protein was also observed in an endoplasmic reticulum (ER) fraction and was included in an MAM fraction.

Based on above results, immunofluorescence analysis was performed using the same DISC1 antibody as that used in the immunoblotting, and as a result, as illustrated in FIG. 1B, it was confirmed that the endogenous DISC1 protein was partially dispersed in cerebral cortical neurons, but was prominently located at the same position as that of ER-RFP and Mito-Crimson.

Moreover, as a result of conducting the same experiment using the DISC1 protein in which the residues 1-201 were deleted (DISC1^{Δ1-201}), as illustrated in FIG. IC, it was confirmed that levels of the DISC1 protein showed along with ER and mitochondria markers were significantly reduced in cerebral cortical neurons. Through these results, it was seen that the MAM localization of the DISC1 protein was mainly governed by the residues 1-201 of the DISC1 protein. The above results were also confirmed from the result illustrated in FIG. 1D, showing that the DISC1^{Δ1-201} protein was barely detected in the crude MAM fraction (MAM+Mito) as compared to the WT DISC1 protein.

### Example 3. Verification of Interaction Between DISC1 and IP₃R1 at MAM and Inhibition of Ligand Binding of IP₃R1 by DISC1

3-1. Verification of Interaction Between DISC1 and IP₃R1 at MAM

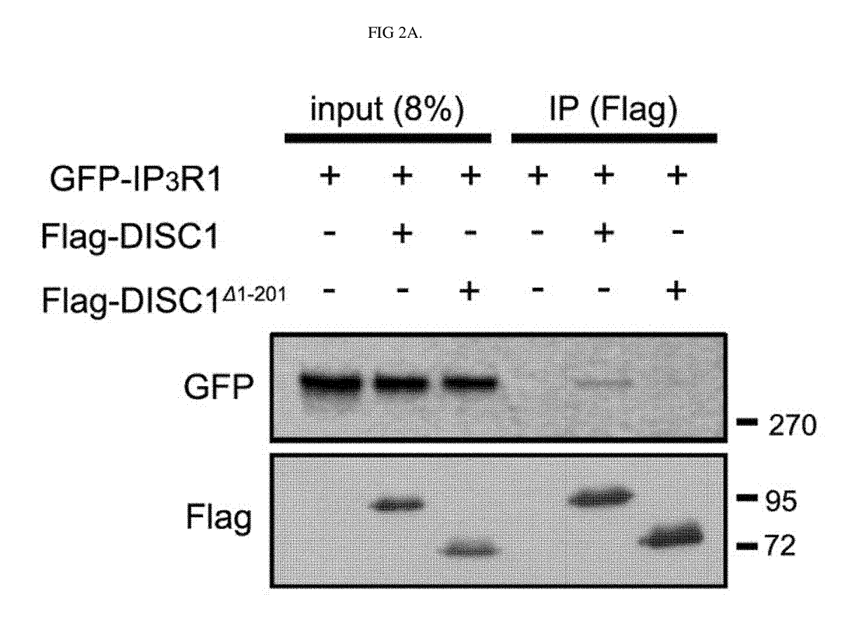

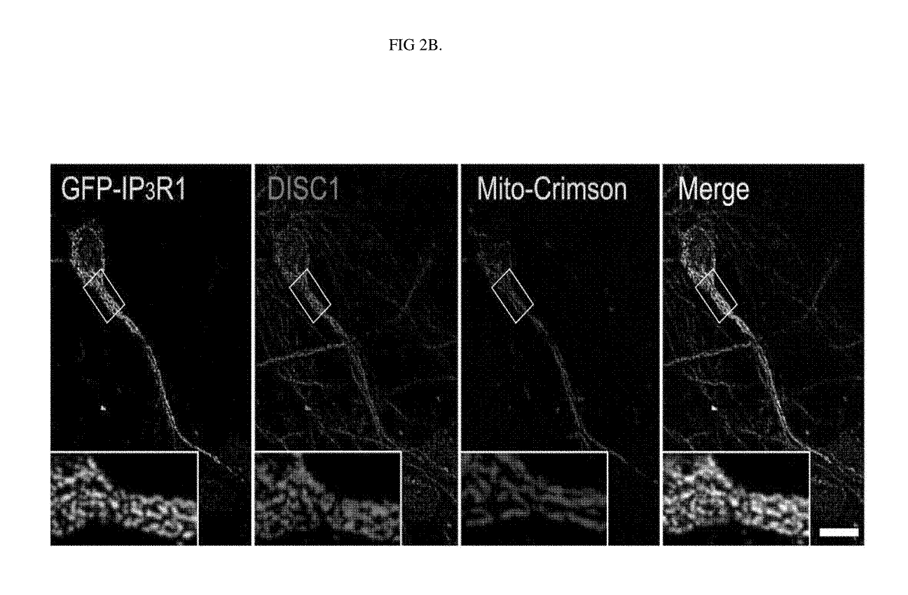

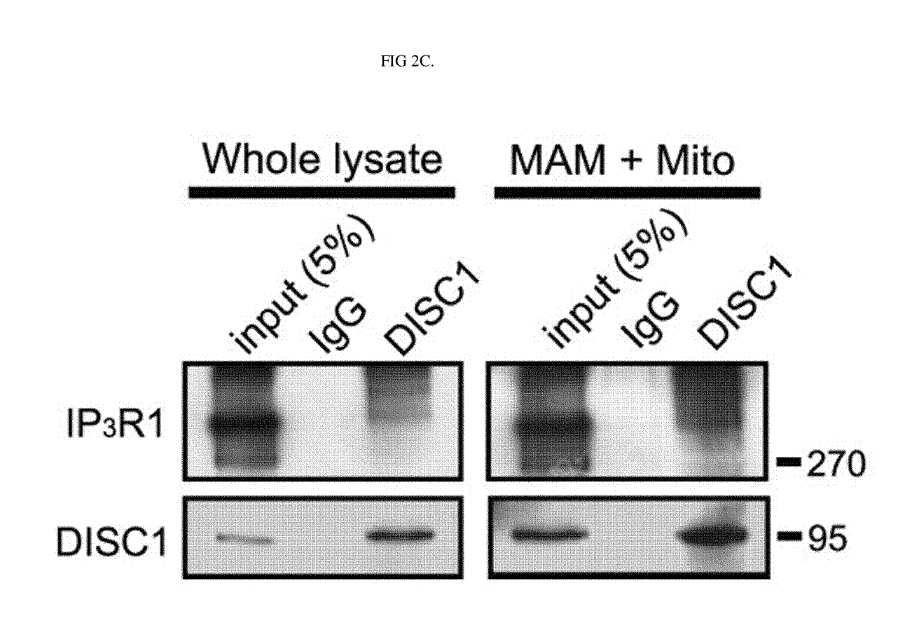

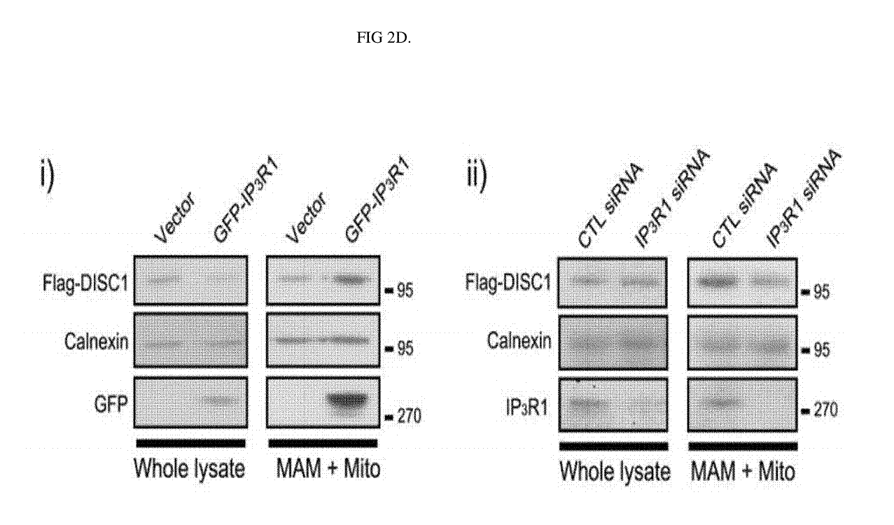

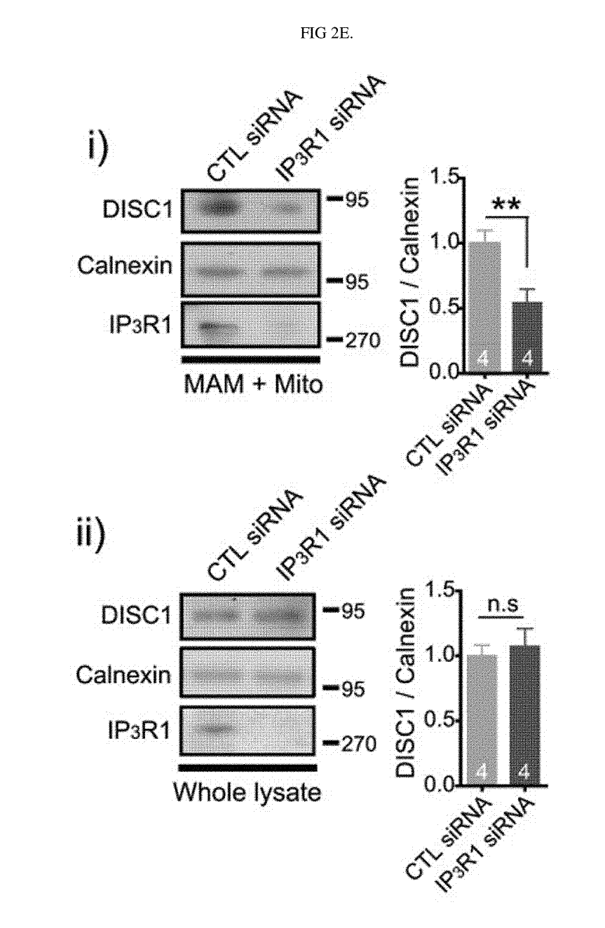

IP₃R1 is known to be predominantly expressed in the brain and abundant in the MAM. Therefore, the MAM localization of DISC1 was confirmed through Example 2, and to examine the interaction between DISC1 and IP₃R1, the inventors of the present disclosure performed immunoprecipitation analysis on the two proteins. As a result, as illustrated in FIG. 2A, it was confirmed that IP₃R1 (GFP-IP3R1) showed a strong interaction with wild-type DISC1 (Flag-DISC1), while not showing such an interaction with DISC1^{Δ1-201}. In addition, as a result of immunofluorescence analysis, as illustrated in FIG. 2B, the endogenous DISC1 protein showed colocalization with GFP-IP₃R1 at contact regions with mitochondria, which were marked by Mito-Crimson, in cerebral cortical neurons. In addition, as illustrated in the immunoblotting results of FIG. 2C, it was confirmed that the endogenous DISC1 protein and the IP₃R1 protein were expressed together in whole lysates and the crude MAM fraction ((MAM+Mito), which was derived from the brains of adult mice.

Moreover, to examine the effect of IP₃R1 on the MAM localization of DISC1, changes in the DISC1 protein in the crude MAM fraction (MAM+Mito) according to a change in expression level of IP₃R1 were analyzed. As a result, it was confirmed that, as illustrated in FIG. 2D, when IP₃R1 was overexpressed (GFP-IP₃R1), the expression of DISC1 (Flag-DISC1) was significantly increased in the crude MAM fraction, whereas, when the expression of IP₃R1 was inhibited by treatment with IP₃R1 siRNA, as illustrated in FIGS. 2D and 2E, both the levels of Flag-DISC1 and the endogenous DISC1 protein were significantly reduced. However, unlike the results shown in the crude MAM fraction, there was no change in the amount of the DISC1 protein in the whole lysates.

The above results suggest that IP₃R1 plays an important role in the MAM localization of DISC1.

3-2. Identification of Inhibition of Ligand Binding of IP₃R1 by DISC1

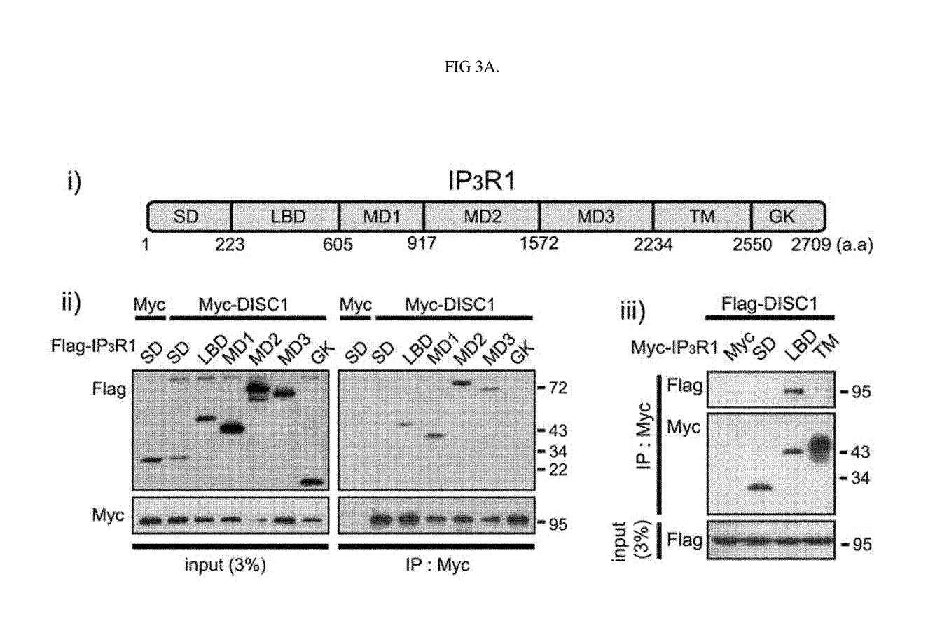

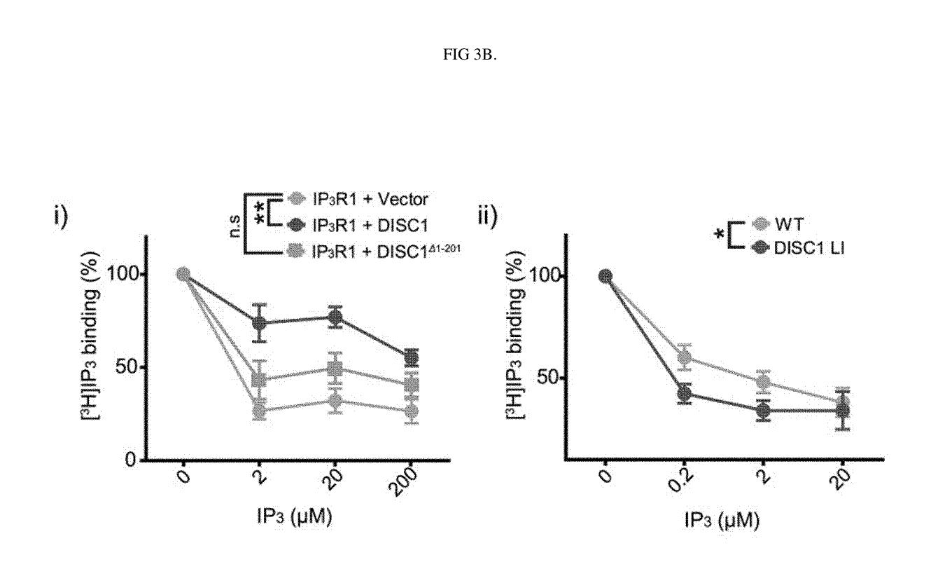

To identify an IP₃R1 domain that binds to DISC1 based on the above results, expression constructs for functional domains of IP₃R1, as illustrated in FIG. 3A, were produced, and it was analyzed through immunoprecipitation whether or not DISC1 interacted with each domain. As a result, it was confirmed that DISC1 interacted with the remaining domains other than suppressor (SD), gate-keeping (GK), and transmembrane (TM) domains, i.e., a ligand-binding domain (LBD) and modulatory domains (MD1, MD2, and MD3).

In IP₃R1, the ligand-binding and modulatory domains are critical regions for ligand binding of IP₃Rs, and thus the inventors of the present disclosure examined the influence of DISC1 on binding between IP₃R1 and its ligand, i.e., IP₃. For this, competitive IP₃ binding analysis was performed using IP3R1, DISC1, or DISC1^{Δ1-201}, which was isolated from HEK293FT cells by immunoprecipitation with antibodies according to the methods of Examples 1 to 3. As a result, as illustrated in FIG. 3B, it was confirmed that unlike DISC1^{Δ1-201}, in the case of DISC1, the binding ability of unlabeled IP₃ relative to [³H]IP₃ bound to IP₃R1 was decreased. In addition, as a result of conducting the same experiment as described above using the endogenous IP₃R1 and DISC1 proteins isolated from wild-type (WT) mice or mice with an impaired DISC1 locus (DISC1 LI), it was confirmed that unlabeled IP₃ binding to IP₃R1 was significantly increased in DISC1 LI-derived samples, as compared to the WT mice.

These results indicate that DISC1 inhibits ligand binding of IP₃R1.

### Example 4. Identification of Role of DISC1 in ER-Mitochondria Ca²⁺ Transfer Through the MAM

4-1. Verification of ER-Mitochondria Ca²⁺ Transfer Regulation Mediated by DISC1 at the MAM

By considering the functional association between IP₃R1 and DISC1 at the MAM, which was verified in the above example, the inventors of the present disclosure examined whether or not DISC1 was involved in regulating ER-mitochondria Ca²⁺ transfer. For this, expression constructs for GCaMP6, which is a genetically encoded Ca²⁺ indicator, were modified by combining the target sequences for mitochondria or the ER. Subsequently, it was confirmed that the organelle-specific GCaMP6 constructs were expressed in cerebral cortical neurons. In the case of ER Ca²⁺ measurement, the results were verified using ER-GCaMP3, which has a relatively low affinity for Ca²⁺.

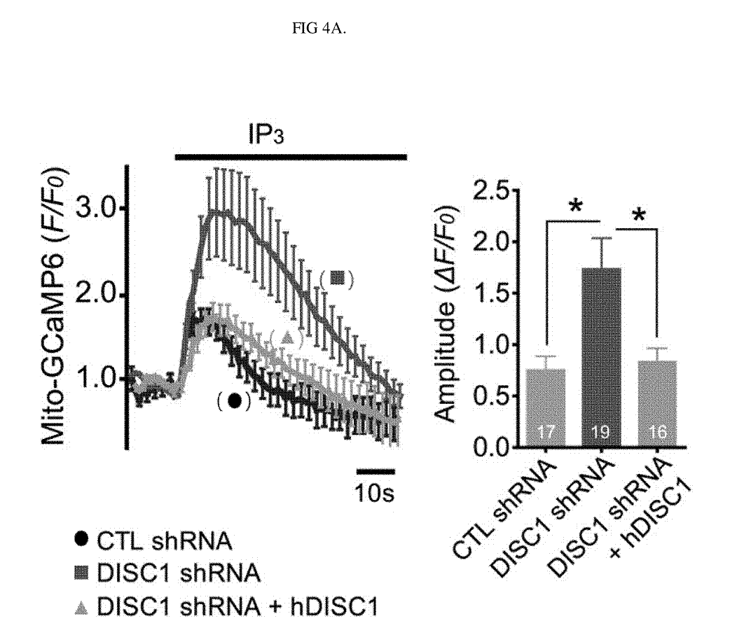

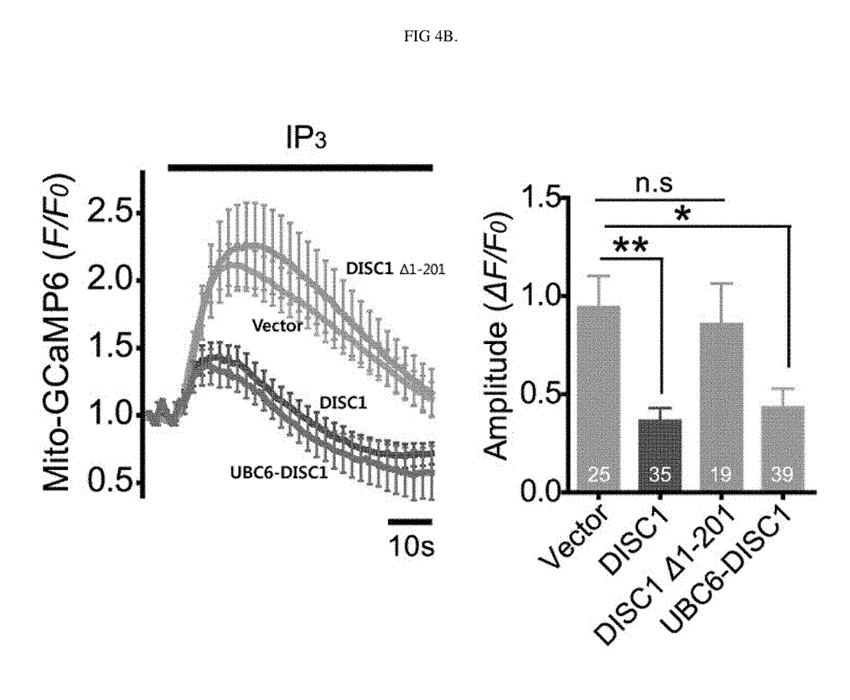

To enhance cell membrane permeabilization, cerebral cortical neurons were preincubated with digitonin and a Ca²⁺-free form of ionomycin in an EGTA- and Ca²⁺-free buffer for 2 minutes, and then washed to prevent the collapse of membranes of other organelles. Subsequently, it was confirmed that this membrane permeabilization process did not affect basal Ca²⁺ levels or general depolarization by the activation of L-type Ca²⁺ channels in neurons. After the permeabilization of the neurons were enhanced using the above method, the neurons were treated with IP₃, and as a result, as illustrated in FIG. 4A, it was confirmed that an IP₃-dependent increase in mitochondrial Ca²⁺ significantly became higher in neurons in which DISC1 expression was inhibited (DISC1 shRNA) than in control neurons (CTL shRNA), and the increase in Ca²⁺ levels was restored similarly to the control, upon overexpressing human DISC1 resistant to shRNA (DISC1 shRNA+hDISC1). In addition, it was confirmed that the levels of Ca²⁺ stored in the ER were dramatically decreased in the neurons in which DISC1 expression was inhibited, upon stimulation with IP₃, and this supports the above results. These results suggest that DISC1 modulates Ca²⁺ release via IP₃R1 on the ER side before Ca²⁺ is transferred into mitochondria. In contrast, as illustrated in FIG. 4B, the overexpression of DISC1 significantly reduced the increase in mitochondrial Ca²⁺ levels induced by IP₃ in permeabilized neurons as compared to that of a control (Vector).

Unlike the above results, when DISC1 expression was inhibited, the increase in mitochondrial Ca²⁺ levels induced by 4-chloro-m-cresol (4-cmc), which is a ryanodine receptor agonist, was unable to be significantly changed, and this indicates that DISC1 is specific to IP₃R-mediated Ca²⁺ transfer.

Moreover, the inhibition of DISC1 expression was shown not to affect mitochondrial capacity for Ca²⁺ uptake in neurons that were preincubated with 2-aminoethyl diphenylborinate (2-APB), which is a selective IP₃R blocker. To further examine the intrinsic capacity of mitochondrial Ca²⁺ uptake, an in vitro mitochondrial Ca²⁺ assay was carried out using pure mitochondrial fractions derived from the brains of WT or DISC1 LI mice with an impaired DISC1 locus. In response to Ca²⁺ pulses in extraocular muscles, fluorescence signals of CaGreen-5N, i.e., an intracellular permeable Ca²⁺ dye, were increased, but were reduced by mitochondrial Ca²⁺ uptake after reaching their peaks. In addition, the rates of decrease, which represent mitochondrial Ca²⁺ uptake rates, were not significantly different between WT and DISC1 LI. These results suggest that the effects of DISC1 on ER-mitochondria Ca²⁺ transfer are not due to changes in the mitochondrial capacity for Ca²⁺ uptake.

To further examine the association between the MAM localization of DISC1 and the regulation of ER-mitochondria Ca²⁺ transfer, the inventors of the present disclosure prepared a DISC1 expression construct (UBC6-DISC1) that was fused with a targeting sequence of yeast UBC6, which is an ER membrane protein, to target DISC1 on the ER/MAM, and confirmed ER and MAM localization of DISC1 in neurons. As illustrated in FIG. 4B, it was confirmed that neurons expressing the construct (UBC6-DISC1) significantly reduced IP₃-mediated Ca²⁺ transfer in a manner similar to the case of the overexpression of WT DISC1, whereas DISC1^{Δ1-201} failed to exhibit significant changes in Ca²⁺ transfer. In addition, DISC1 was predominantly expressed at the outer mitochondrial membrane or in the mitochondrial internal space by recombining the anchoring sequence of mouse AKAP1 or the yeast MIA40 leader sequence, and failed to change the IP₃-dependent mitochondrial Ca²⁺ response.

4-2. Verification of Regulation of ER-Mitochondria Ca²⁺ Transfer of MAM, Mediated by DISC1

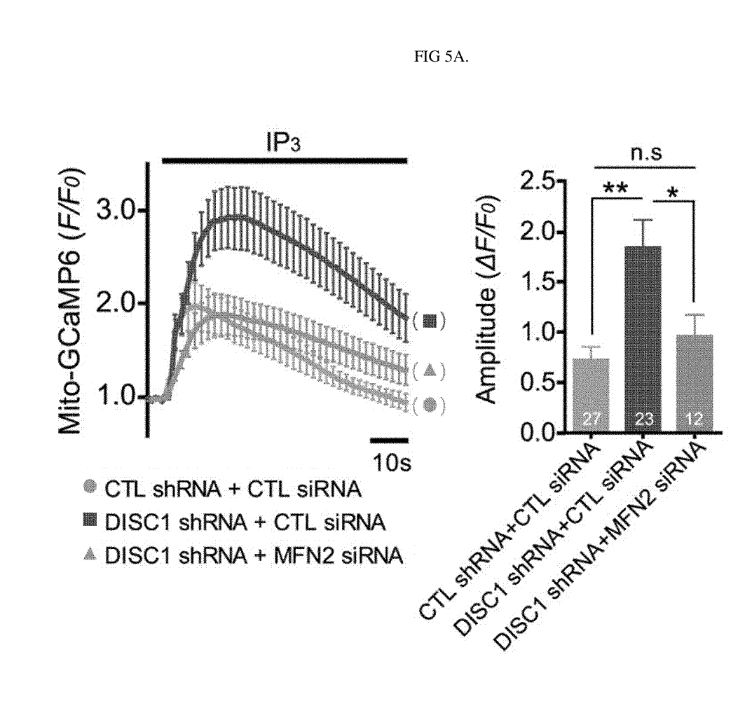

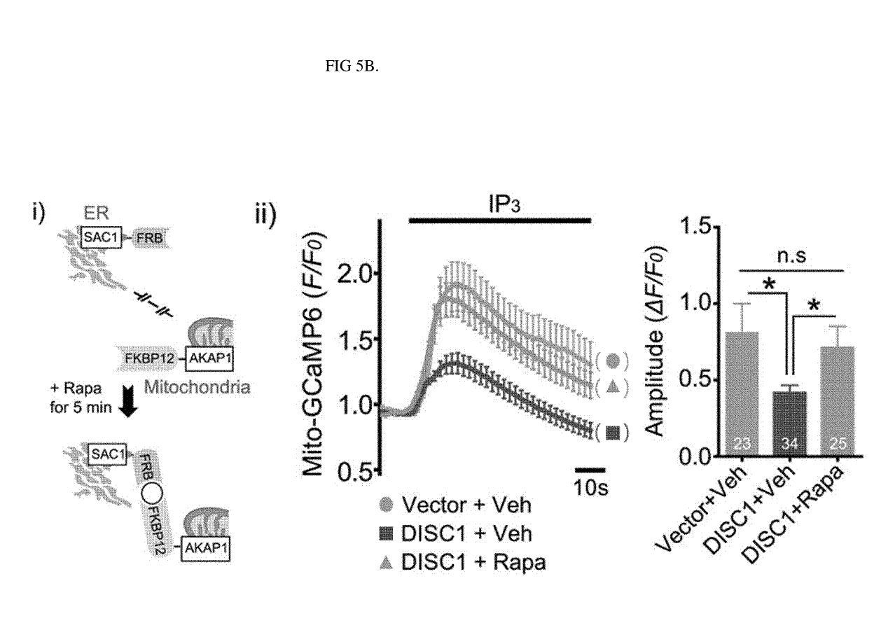

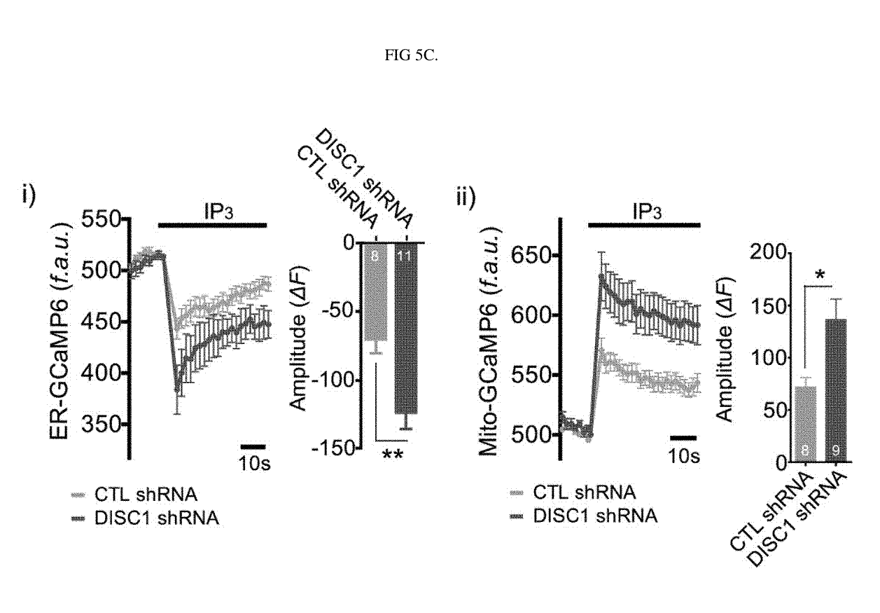

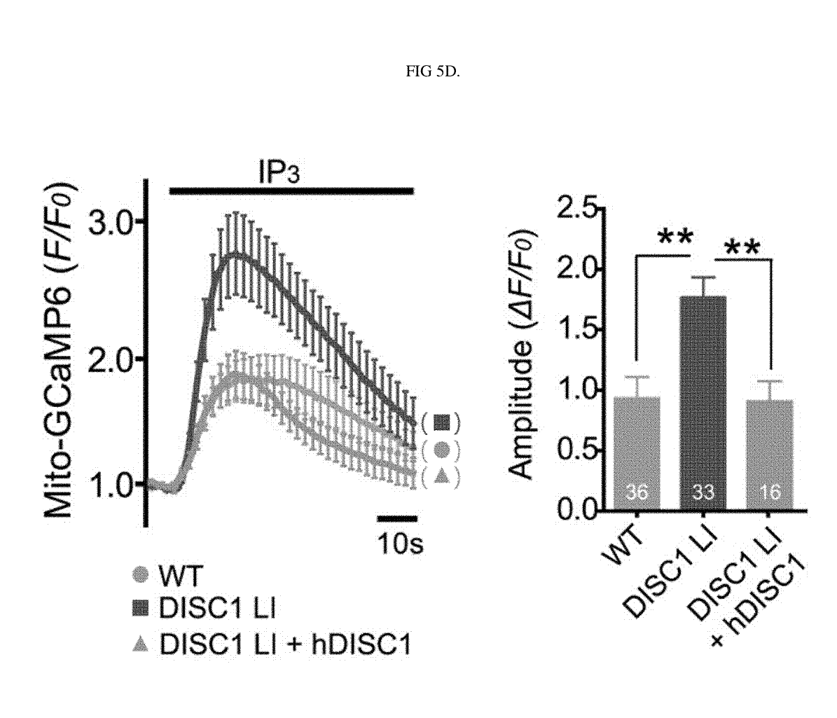

Next, to investigate whether or not the ER-mitochondria Ca²⁺ transfer regulated by DISC1 is controlled by changes in MAM formation, mitofusin 2 (MFN2), which is a protein tethering the ER to mitochondria at the MAM, was used. As a result of an experiment, as illustrated in FIG. 5A, the accumulation of mitochondrial Ca²⁺ was significantly increased in neurons in which DISC1 expression was inhibited (DISC1 shRNA+CTL siRNA) as compared to a control (CTL shRNA+CTL siRNA), similarly to the previous results, and in neurons in which MFN2 expression was inhibited together with the inhibition of DISC1 expression, MAM contact was decreased and the accumulation of mitochondrial Ca²⁺ was decreased to a level similar to the control.

In addition, unlike this, a rapamycin-inducible bridge-forming module (RiBFM), which enables the enhancement of ER-mitochondria contact in response to rapamycin treatment, was used. In particular, two rapamycin-binding domains that localize to the ER and mitochondria, respectively were dramatically merged after treatment with rapamycin for 5 minutes, and after treatment with rapamycin, it was confirmed that the merging lasted for 1 hour even after rapamycin was removed. In addition, it was confirmed that the activation of this module by rapamycin increased ER-mitochondria Ca²⁺ transfer in neurons, as reported previously. As a result of an experiment, as illustrated in FIG. 5B, it was confirmed that the rates of increase in mitochondrial Ca²⁺ accumulation according to IP₃ treatment was decreased in neurons in which the DISC1 protein was overexpressed (DISC1) as compared to a control (Vector+Veh), whereas the dramatic enhancement of ER-mitochondria contact by this module upon rapamycin treatment increased the mitochondrial Ca²⁺ accumulation to a level similar to the control. These results collectively suggest that DISC1-regulated ER-mitochondria Ca²⁺ transfer occurs mainly at the MAM.

Moreover, to more directly verify Ca²⁺ transfer through the MAM under the control of DISC1, in vitro Ca²⁺ analysis was performed. For this, crude MAM fractions (mitochondria-attached MAM) were isolated from neuroblastoma Cath.-a-differentiated (CAD) cells transfected with GCaMP6s and shRNA. As a result of an experiment, as illustrated in FIG. 5C, it was confirmed that the crude MAM fraction isolated from the cells in which DISC1 expression was inhibited showed a greater reduction in MAM Ca²⁺ upon IP₃ treatment, as compared to a control, whereas mitochondrial Ca²⁺ levels were dramatically increased as compared to the control. In addition, as illustrated in FIG. 5D, it was confirmed that cerebral cortical neurons of the DISC1 LI embryos with an impaired DISC1 locus showed a greater increase in ER-mitochondria Ca²⁺ transfer than that in a control, similarly to the case of the inhibition of DISC1 expression, and these results showed significant levels in the control by hDISC1 expression.

Taken together, the above results indicate that DISC1 dysfunction may cause abnormal ER-mitochondria Ca²⁺ transfer at the MAM.

### Example 5. Regulation of Oxidative Stress-Dependent ER-Mitochondria Ca²⁺ Transfer by DISC1

Recent studies have suggested that susceptibility to oxidative stress underlies neuronal environments associated with the pathophysiology of schizophrenia. Intriguingly, oxidative stress induces gradual Ca²⁺ release from the ER and Ca²⁺ transfer into mitochondria in various types of cells, including neurons. Based on these results, the inventors of the present disclosure examined whether or not DISC1 affects ER-mitochondria Ca²⁺ transfer induced by oxidative stimuli.

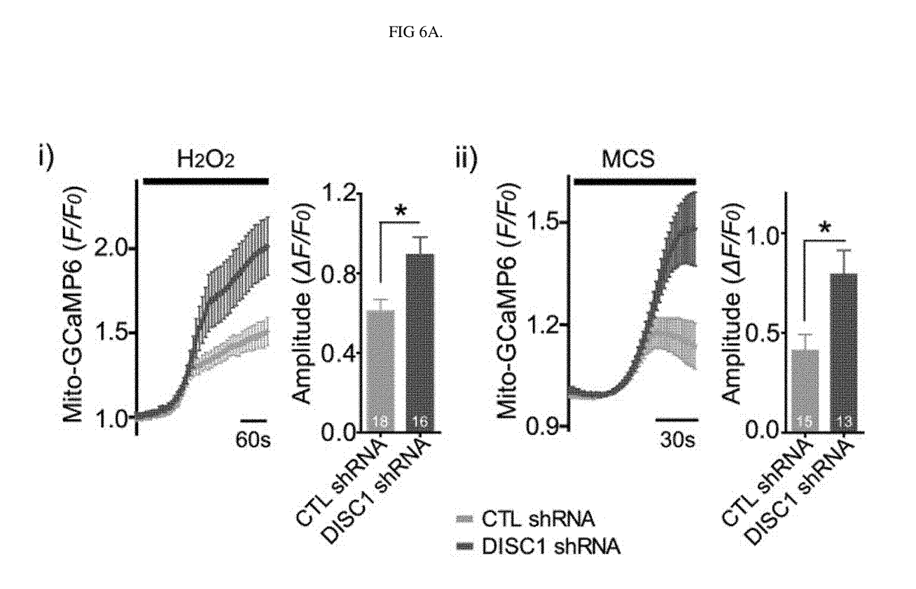

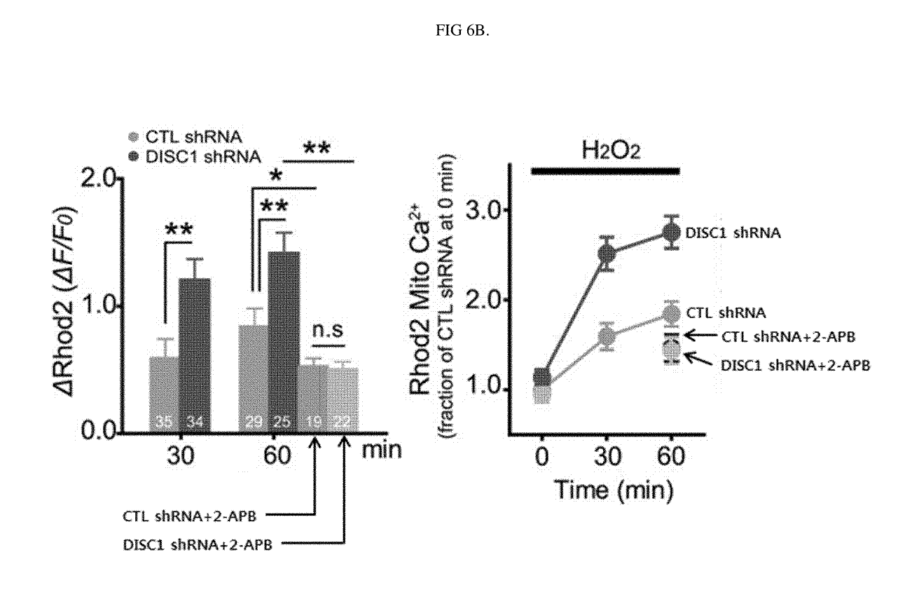

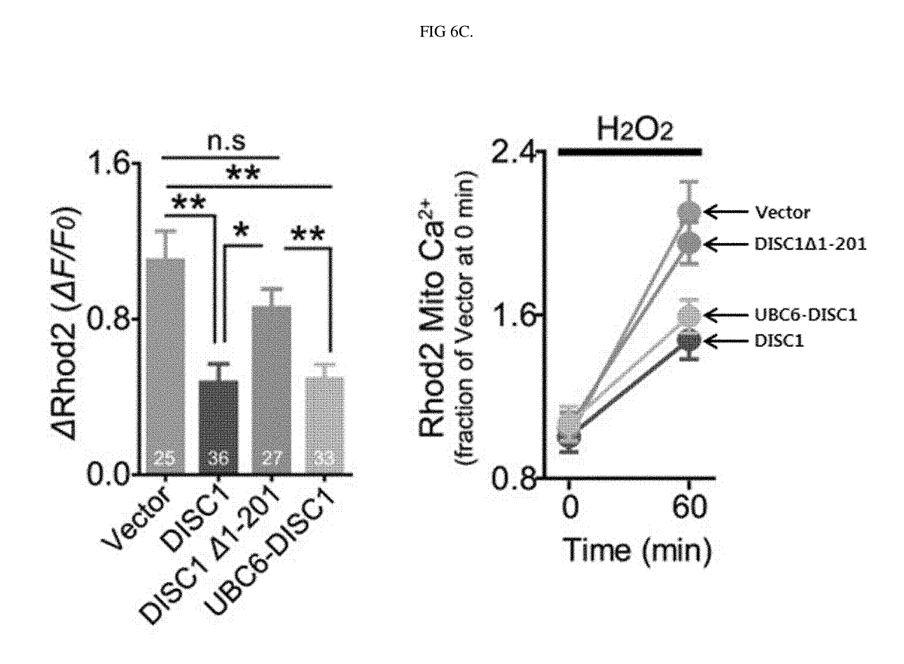

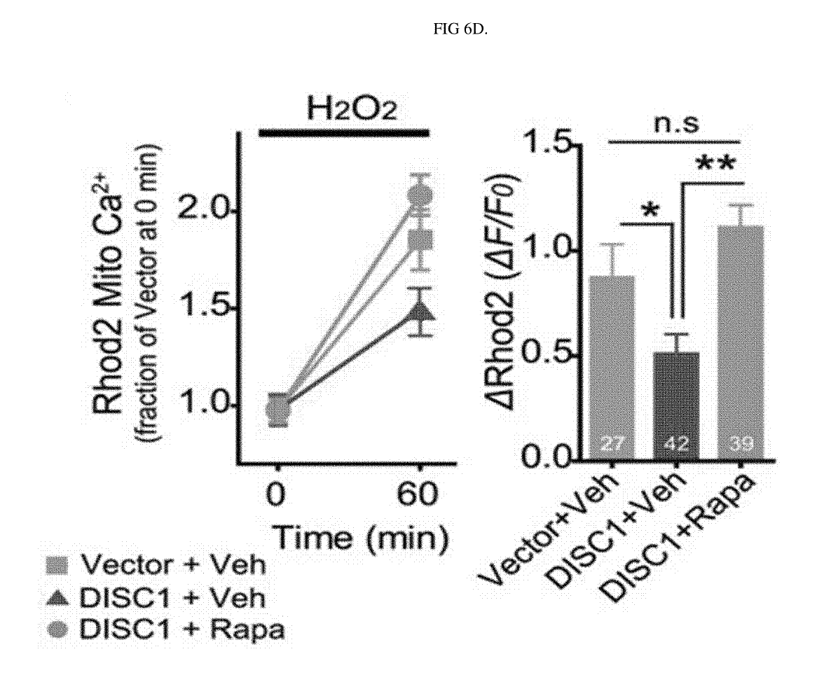

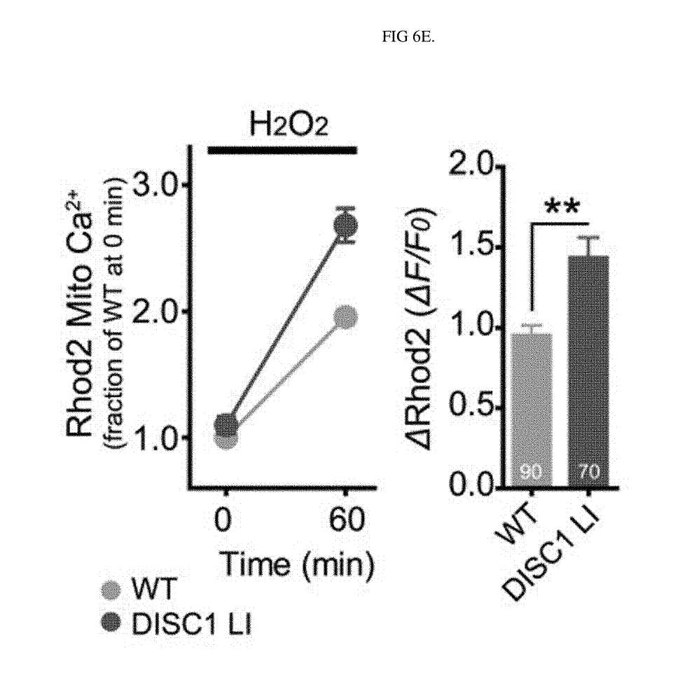

As a result, as illustrated in FIG. 6A, control neurons (CTL shRNA) displayed a smaller and slower increase in mitochondrial Ca²⁺ upon treating the cerebral cortical neurons with H₂O₂, as compared to the case of IP₃ treatment, and neurons in which DISC1 expression was inhibited (DISC1 shRNA) showed a significantly dramatic increase in mitochondrial Ca²⁺ levels. In addition, even in the case of treatment with mercaptosuccinic acid (MSC), which is an inhibitor of glutathione peroxidase that endogenously generates H₂O₂, the neurons in which DISC1 expression was inhibited showed a more dramatic increase in mitochondrial Ca²⁺ than that in the control. From these results, it was confirmed that ER Ca²⁺ levels were reduced in the neurons in which DISC1 expression was inhibited (DISC1 shRNA).

In light of the slower increase in mitochondrial Ca²⁺ levels in response to oxidative stress, the inventors of the present disclosure measured mitochondrial Ca²⁺ levels under oxidative stress over a long time period. At this time, to measure mitochondrial Ca²⁺ levels at specific time points during incubation of neurons with H₂O₂, Rhod2/AM, which is a mitochondria-specific chemical Ca²⁺ indicator, was used instead of Mito-GCaMP6 capable of causing variations due to different expression levels of Mito-GCaMP6 at multiple time points. As a result of an experiment, as illustrated in FIG. 6B, mitochondrial Ca²⁺ levels were increased proportional to the incubation time after H₂O₂ treatment, and the neurons in which DISC1 expression was inhibited (DISC1 shRNA) exhibited a significant increase in Ca²⁺ accumulation, but these results were not shown in the case of preincubation with 2-APB, which is a selective IP₃R inhibitor (DISC1 shRNA+2-APB). This indicates that IP₃R plays a vital role in regulating ER-mitochondria Ca²⁺ transfer induced by oxidative stress.

In contrast, as illustrated in FIG. 6C, the overexpression of WT DISC1 and UBC6-DISC1 significantly reduced a degree to which mitochondrial Ca²⁺ levels were increased, at 1 hour after H₂O₂ incubation, as compared to the control, and such a reduction effect was not observed in DISC1^{Δ1-201}. In addition, as illustrated in FIG. 6D, it was confirmed that mitochondrial Ca²⁺ accumulation in response to oxidative stress was increased in a case (DISC1+Rapa) in which ER-mitochondria contact was enhanced by RiBFM, and this was seen to offset the effect of DISC1 overexpression on oxidative stress-induced mitochondrial Ca²⁺ accumulation. Consistent with these results, as illustrated in FIG. 6E, it was confirmed that the cerebral cortical neurons derived from the DISC1 locus-impaired mouse embryos (DISC1 LI) exhibited a significant increase in mitochondrial Ca²⁺ levels similarly to the case of the inhibition of DISC1 expression upon incubation with H₂O₂, as compared to wild-type neurons (WT).

### Example 6. Regulation of Oxidative Stress-Mediated Dysfunction by DISC1 in Mitochondria

Excessive Ca²⁺ accumulation in mitochondria has been reported to deregulate the activity of the mitochondrial electron transfer chain, causing the collapse (depolarization) of mitochondrial membrane potential and promotion of ROS generation. Such mitochondrial dysfunction was observed in patients with schizophrenia and in animal models that display the phenotypes of schizophrenia, and this implies that the deterioration of mitochondrial activity may be a component of schizophrenia pathophysiology. Therefore, the inventors of the present disclosure examined that the influence of DISC1 on oxidative stress-mediated mitochondrial dysfunction in cerebral cortical neurons. More particularly, to measure changes in mitochondrial membrane potential in response to oxidative stress, neurons were preincubated with tetramethylrhodamine methyl ester perchlorate (TMRM), which is a chemical indicator of mitochondrial potential, and exposed to H₂O₂.

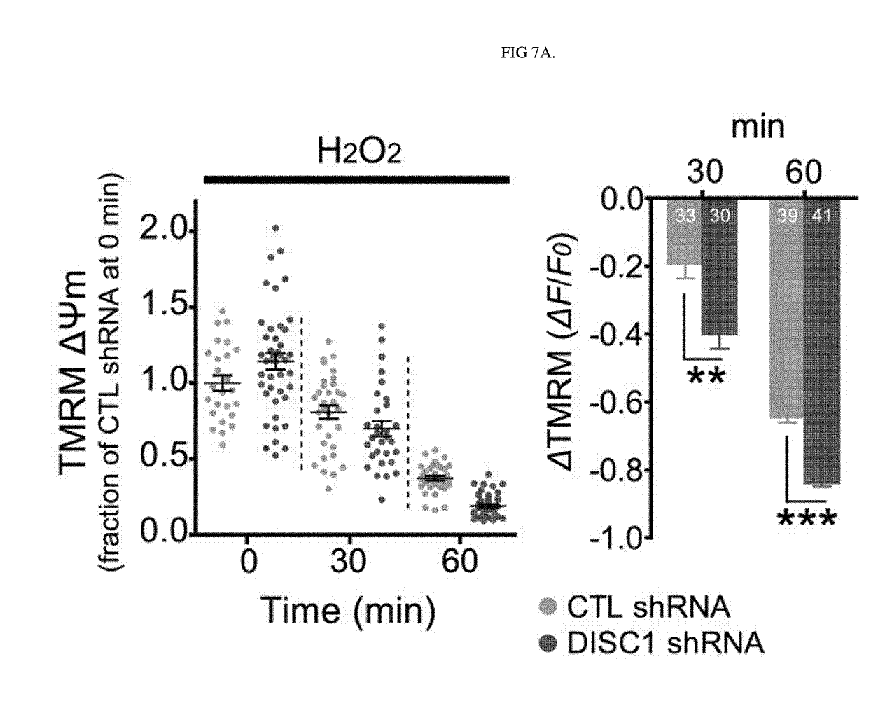

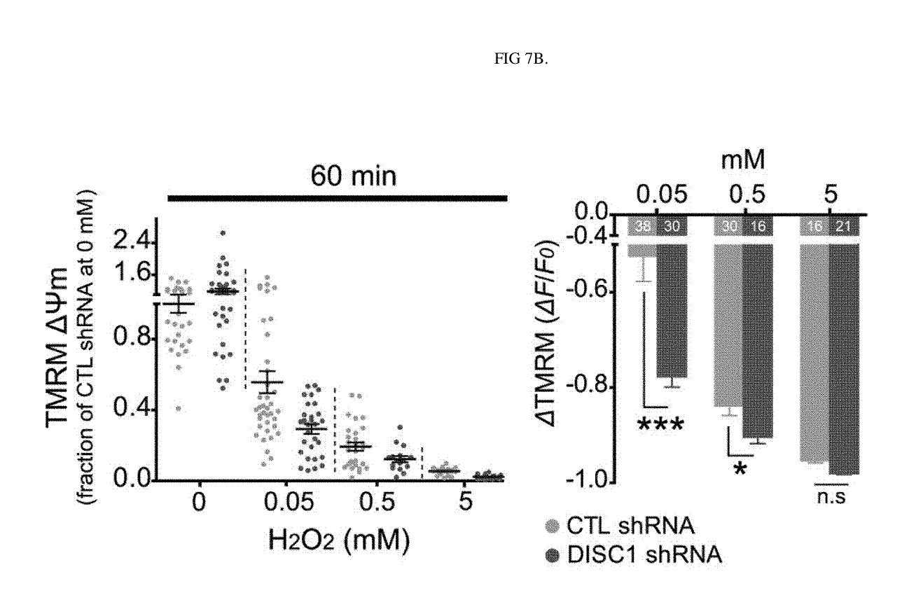

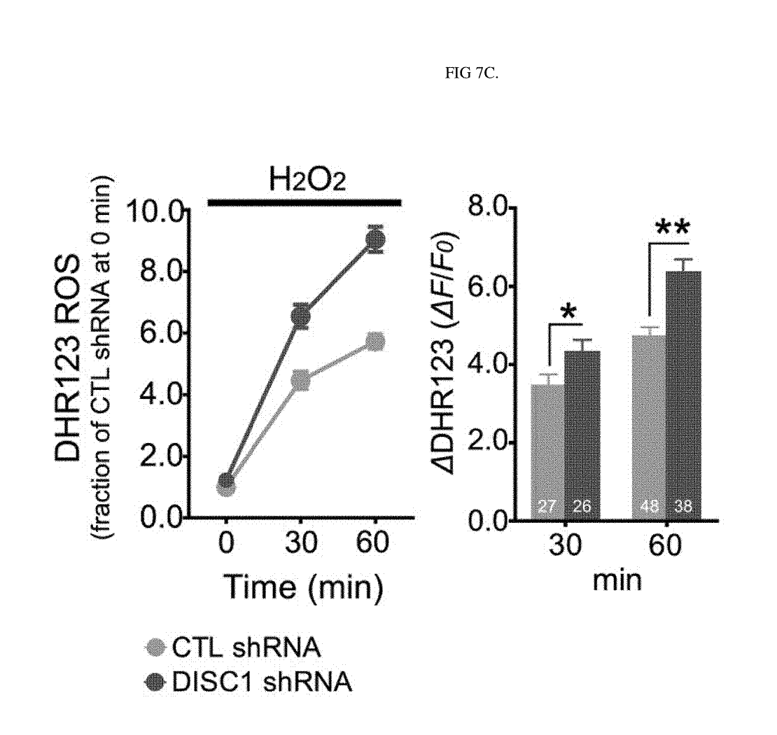

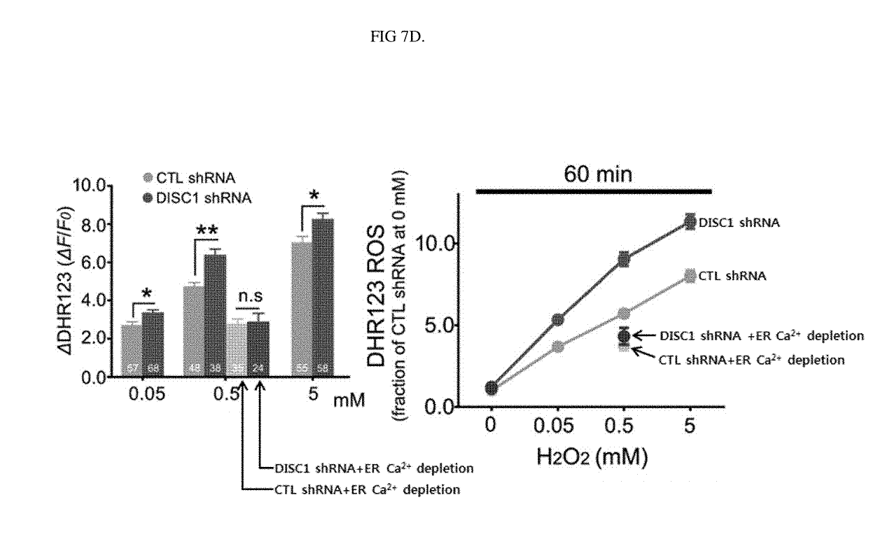

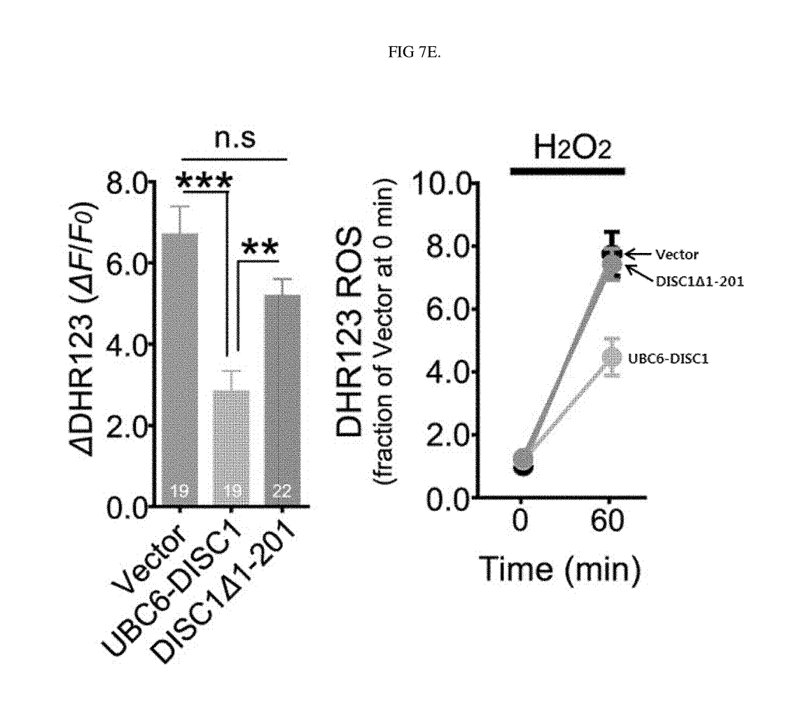

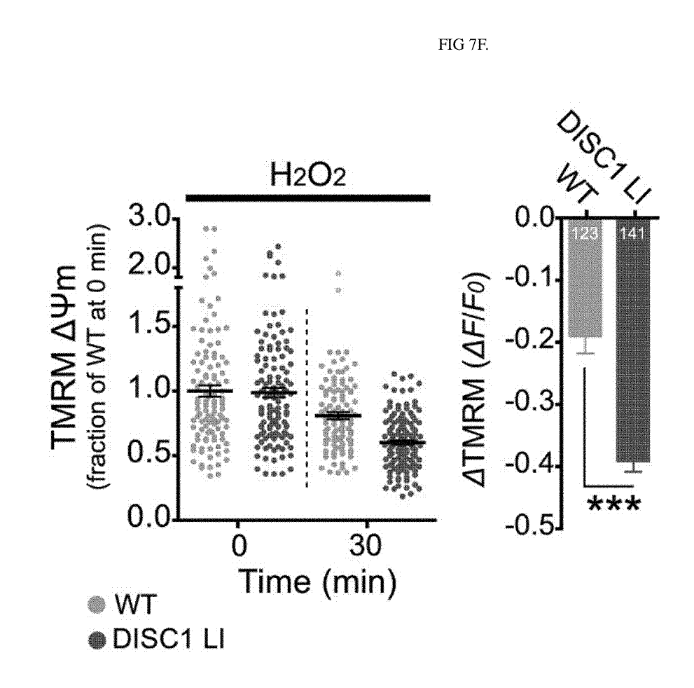

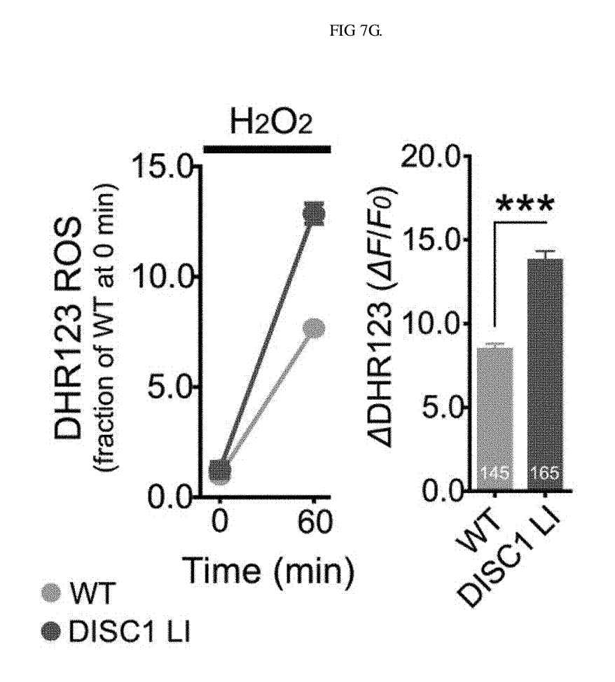

As a result, as illustrated in FIGS. 7A and 7B, it was observed that in neurons in which DISC1 expression was inhibited (DISC1 shRNA), H₂O₂-induced collapse of mitochondrial membrane potential was accelerated in an exposure time- and treatment dose-dependent manner, as compared to the control neurons (CTL shRNA). Consistent with this, as a result of measuring the degree of ROS generation by dihydrorhodamine-123 (DHR-123), which is an indicator of mitochondrial ROS, in the same experiment, as illustrated in FIGS. 7C and 7D, an increase in ROS generation was accelerated in the neurons in which DISC1 expression was inhibited (DISC1 shRNA) in a time- and dose-dependent manner. In addition, in the case of the pre-depletion of ER-stored Ca²⁺, H₂O₂-dependent ROS production was reduced in both a control (CTL shRNA+ER Ca²⁺ depletion) and neurons in which DISC expression was inhibited (DISC1 shRNA+ER Ca²⁺ depletion), and it was confirmed that differences in ROS generation between the control and the DISC1 knockdown neurons were eliminated. Moreover, in the case of DISC1 overexpression, as illustrated in FIG. 7E, UBC6-DISC1 significantly reduced ROS production in response to H₂O₂, whereas DISC1^{Δ1-201} failed to exhibit a significant difference as compared to a control vector. Consistent with these results, as illustrated in FIGS. 7F and 7G, DISC1 LI embryo-derived neurons displayed greater changes in mitochondrial membrane potential and ROS production as a result of incubation with H₂O₂ as compared to WT neurons.

Taken altogether, these results demonstrate that the function of DISC1 at the MAM is closely associated with mitochondrial functionality during oxidative stress via ER-mitochondria Ca²⁺ transfer.

### Example 7. Verification of Induction of Mitochondrial Ca²⁺ Accumulation by Corticosterone in Oxidative Stress-Dependent Manner

Earlier studies have demonstrated that both acute and chronic treatments with excess amounts of glucocorticoids result in the impairment of oxidative phosphorylation, causing deficits in mitochondrial ATP production, and that a drastic increase in ROS leads to oxidative stress in glucocorticoid receptor-rich brain regions, including the hippocampus and cerebral cortex. Thus, the inventors of the present disclosure hypothesized that excessive glucocorticoids could lead to ER-mitochondria Ca²⁺ transfer by inducing oxidative stress.

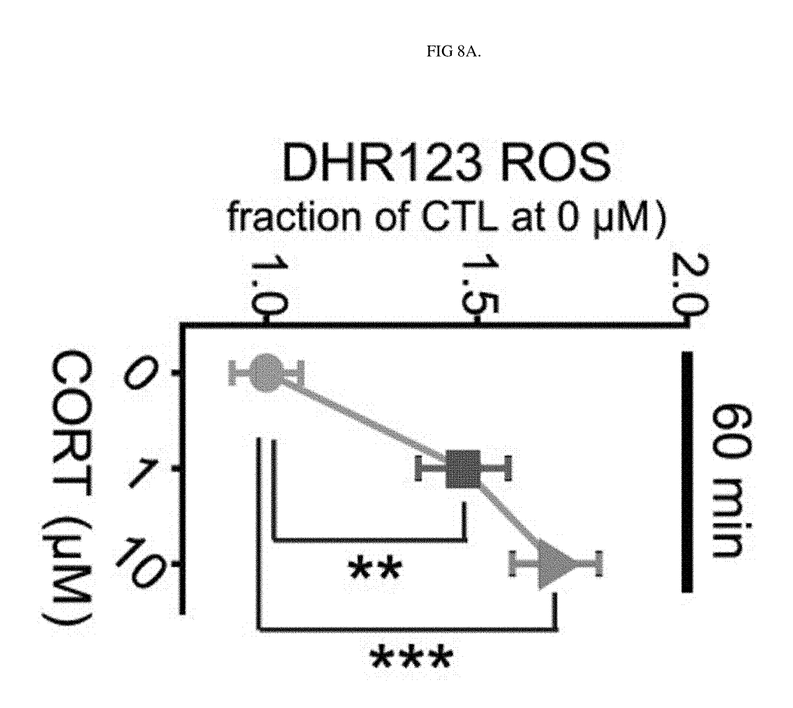

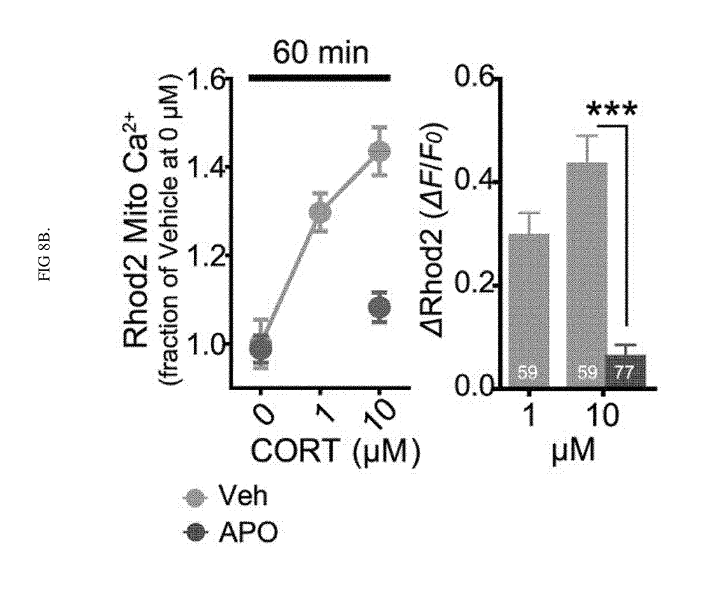

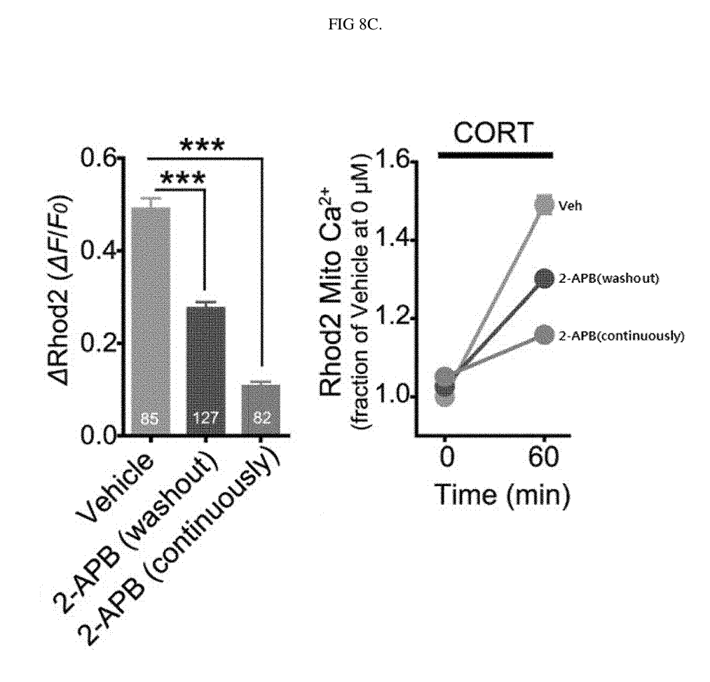

To verify the hypothesis, changes in ROS and mitochondrial Ca²⁺ levels in cerebral cortical neurons were assessed after treatment with corticosterone (CORT), which is a glucocorticoid stress hormone. As a result, as illustrated in FIGS. 8A and 8B, upon treatment with corticosterone for 1 hour, significant increases in ROS and mitochondrial Ca²⁺ levels were observed proportional to the treatment dose. To determine whether this glucocorticoid-dependent increase in mitochondrial Ca²⁺ levels relies on the induction of oxidative stress, apocynin (APO), which is an antioxidant and a ROS scavenger, was used. As a result, as illustrated in FIG. 8B, it was confirmed that in the case of preincubation with APO, the corticosterone-induced increase in mitochondrial Ca²⁺ levels was significantly reduced. Moreover, as illustrated in FIG. 8C, it was confirmed that in the case of preincubation of neurons with 2-APB, which is an IP₃R inhibitor, the corticosterone-induced increase in mitochondrial Ca²⁺ levels was not observed. In contrast, it was confirmed that, when neurons were treated with corticosterone for 1 hour, the capacity of the ER for Ca²⁺ storage or IP₃ generation was not changed.

Taken altogether, these results indicate that ER-mitochondria Ca²⁺ transfer may be regulated by corticosterone interlinked with the induction of oxidative stress.

### Example 8. Verification of Regulation of Corticosterone-Dependent ER-Mitochondria Ca²⁺ Transfer by DISC1

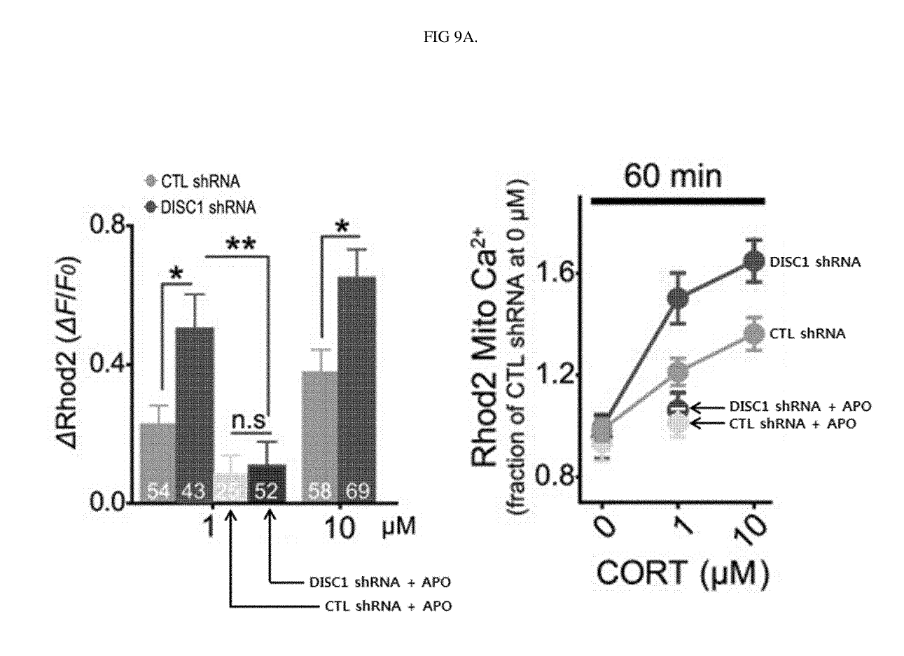

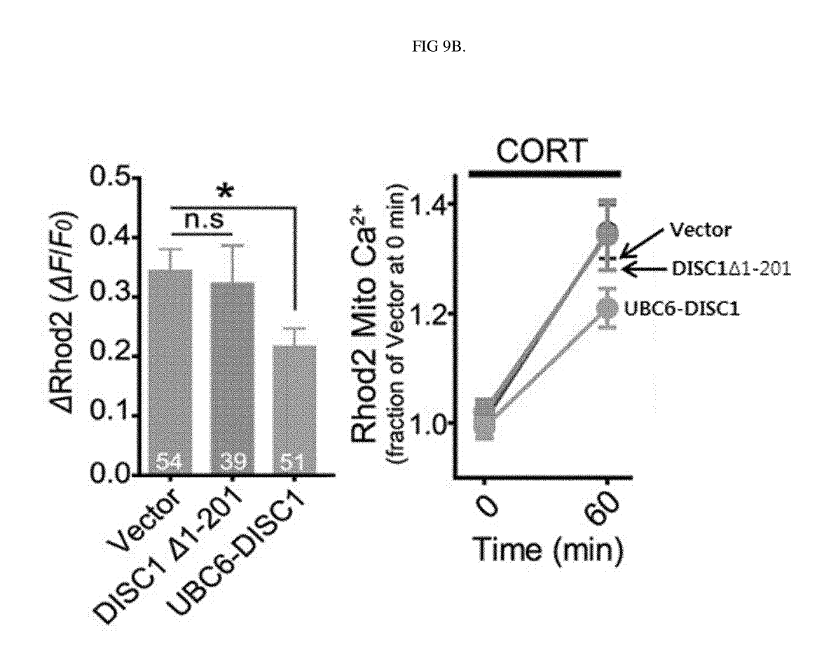

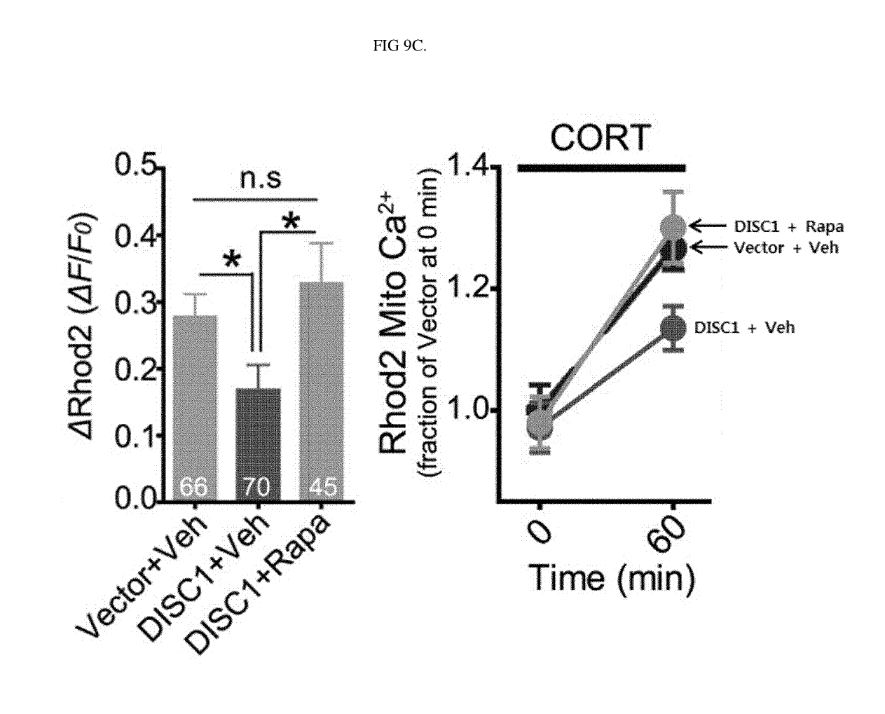

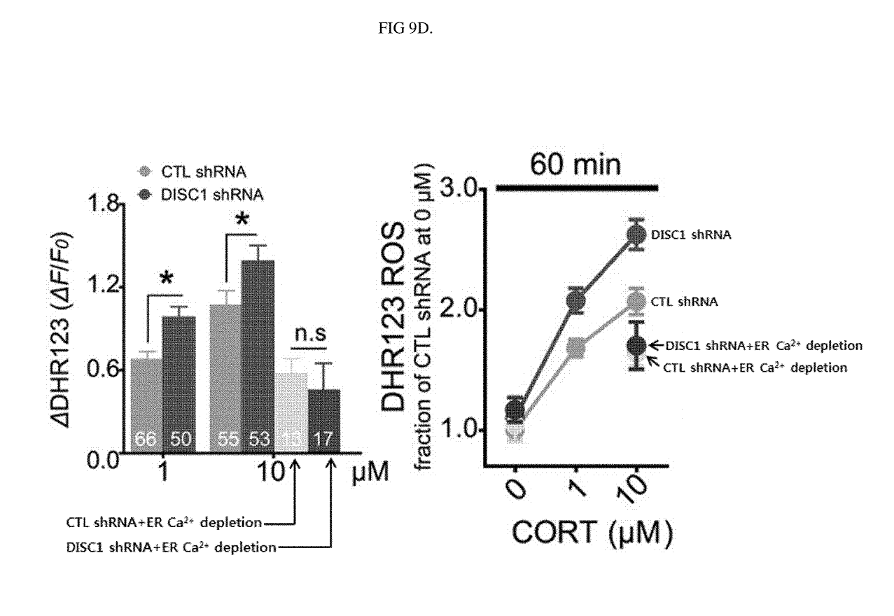

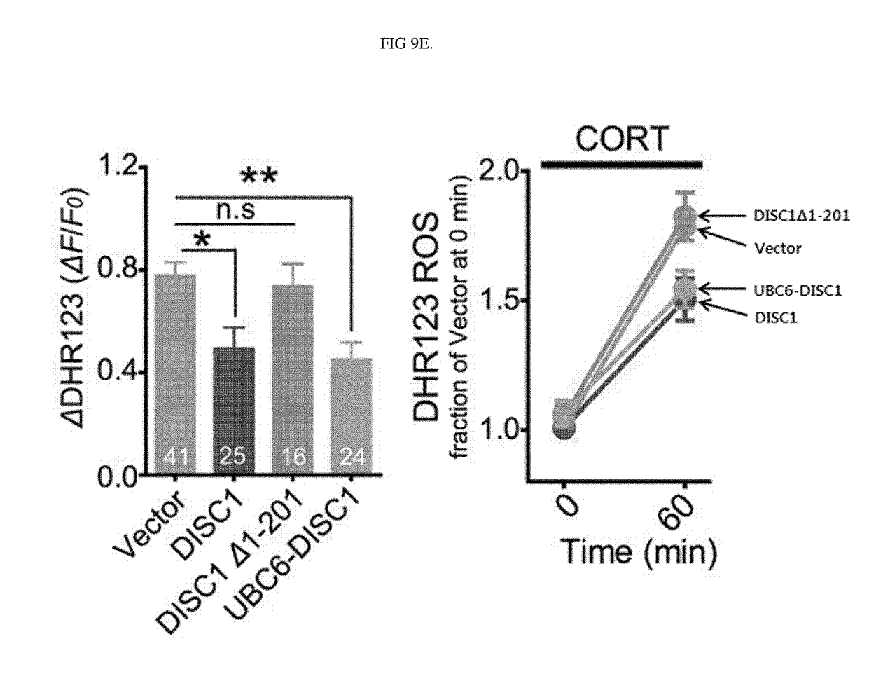

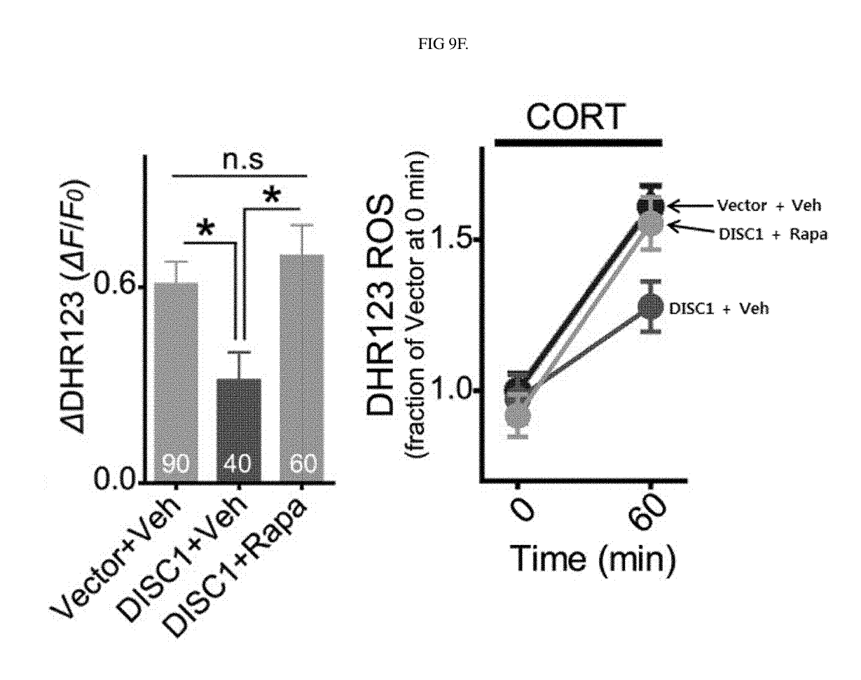

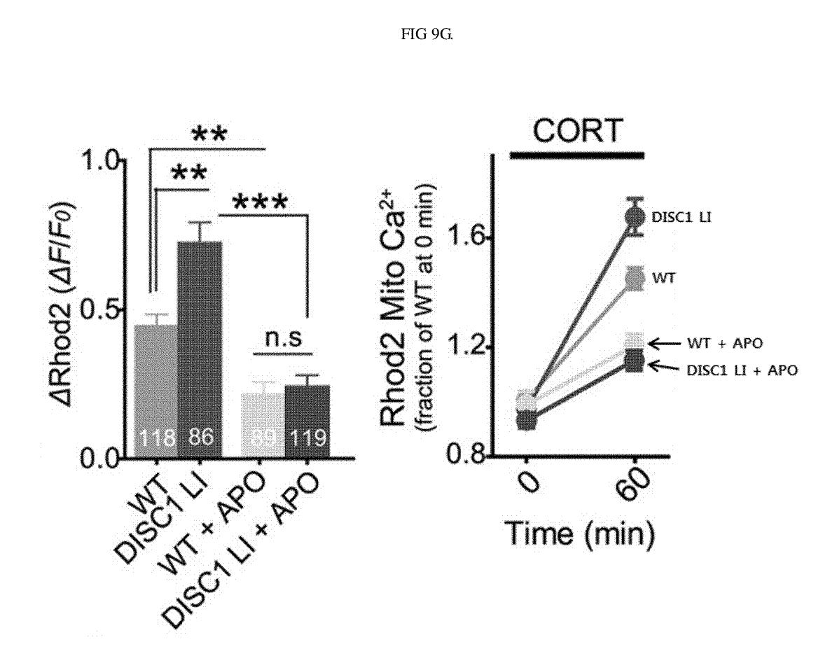

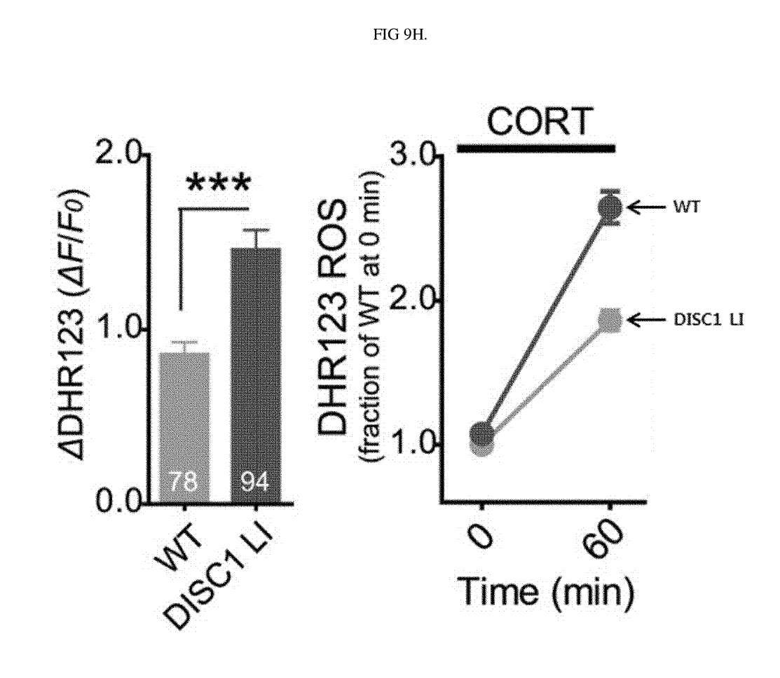

Since it was confirmed through the above examples that DISC1 regulated oxidative stress-induced ER-mitochondria Ca²⁺ transfer, the inventors of the present disclosure further examined whether DISC1 affects corticosterone-induced mitochondrial Ca²⁺ accumulation. As a result, as illustrated in FIG. 9A, it was confirmed that, when treated with corticosterone for 1 hour, neurons in which DISC1 expression was inhibited (DISC1 shRNA) exhibited a significant increase in mitochondrial Ca²⁺ levels, as compared to control neurons (CTL shRNA) and this was significantly reduced by treatment with APO, which is an antioxidant and a ROS scavenger (CTL shRNA+APO and DISC1 shRNA+APO).

In addition, the inventors of the present disclosure further investigated whether the MAM localization of DISC1 affected the corticosterone-induced increase in mitochondrial Ca²⁺ levels. As a result, as illustrated in FIG. 9B, in a case in which neurons overexpressing UBC6-DISC1 (UBC6-DISC1) were incubated with corticosterone, mitochondrial Ca²⁺ accumulation was reduced as compared to control neurons (Vector), whereas DISC1^{Δ1-201} did not exhibit such a reduction effect. Moreover, as illustrated in FIG. 9C, it was confirmed that, in a case in which MAM formation was enhanced by treating the neurons overexpressing DISC1 (DISC1) with rapamycin, the corticosterone-induced mitochondrial Ca²⁺ accumulation was increased again. These results indicate that DISC1 plays a vital role in glucocorticoid-induced ER-mitochondria Ca²⁺ transfer at the MAM.

Next, it was examined whether excessive mitochondrial Ca²⁺ accumulation induced by corticosterone caused excessive ROS generation in neurons in which DISC1 expression was inhibited. As a result, as illustrated in FIG. 9D, the degree of ROS generation was significantly increased in neurons in which DISC1 expression was inhibited (DISC1 shRNA) as compared to control neurons (CTL shRNA), and this result was not observed in neurons in which ER Ca²⁺ was previously depleted (CTL shRNA+ER Ca²⁺ depletion and DISC1 shRNA+ER Ca²⁺ depletion). Moreover, as illustrated in FIG. 9E, it was confirmed that WT DISC1 and neurons overexpressing DISC1 (UBC6-DISC1) reduced ROS levels, which were increased by incubation with corticosterone, whereas DISC1^{Δ1-201} failed to reduce the increased ROS level. In addition, as illustrated in FIG. 9F, the degree of ROS generation was increased again in neurons overexpressing DISC1 when an increase in MAM formation was induced by RiBFM. Under these experimental environments, rapamycin itself did not affect the increase in mitochondrial Ca²⁺ levels and oxidative stress- and glucocorticoid-induced ROS generation. Lastly, as illustrated in FIGS. 9G and 9H, neurons derived from DISC1 LI mouse embryos showed excessive ROS generation and mitochondrial Ca²⁺ accumulation in response to corticosterone similarly to the neurons in which DISC1 expression was inhibited, as compared to a control (WT). Consistent with the results shown in FIG. 9A, it was confirmed that APO treatment significantly reduced the difference in mitochondrial Ca²⁺ accumulation between WT and DISC1 LI neurons.

As is apparent from the foregoing description, as a result of studying the association between DISC1 and psychological stress, the inventors of the present disclosure verified a function of DISC1 in downregulating ER-mitochondria Ca²⁺ transfer induced by stress hormone-mediated oxidative stress by competitively inhibiting binding of IP₃ to inositol 1,4,5-trisphosphate (IP₃) receptor type1 (IP₃R1) by binding to the IP₃R1 at the MAM, and an acting site of DISC1, and this provides a model of intracellular calcium response to physiological stress, and DISC1, a stress modulating substance, and the model can be usefully used in related fields for the prevention and treatment of stress-related diseases.

The above description of the present disclosure is provided for illustrative purposes only, and it will be understood by one of ordinary skill in the art to which the present disclosure pertains that the invention may be embodied in various modified forms without departing from the spirit or essential characteristics thereof. Thus, the embodiments described herein should be considered in an illustrative sense only and not for the purpose of limitation.

What is claimed is:

1. A method for treating a stress-related disease, comprising:
administering to a subject in need thereof an effective amount of disrupted in schizophrenia 1 (DISC1) protein or a gene encoding the DISC1 protein.

2. The method according to claim 1, wherein the stress-related disease is selected from the group consisting of sleep disorders, depression, adaptive disorders, eating disorders, and anxiety disorders.

3. The method according to claim 1, wherein the gene is inserted into a plasmid expression vector or a viral vector.

4. The method according to claim 1, wherein the DISC1 protein regulates endoplasmic reticulum-mitochondria Ca²⁺ transfer induced by stress hormone-mediated oxidative stress at a mitochondria-associated endoplasmic reticulum membrane (MAM).

5. The method according to claim 4, wherein the DISC 1 protein regulates Ca²⁺ transfer by competitively inhibiting binding of IP₃ to inositol 1,4,5-trisphosphate (IP₃) receptor type1 (IP₃R1) by binding to the IP₃R1 at the MAM.

6. The method according to claim 4, wherein the stress hormone comprises a glucocorticoid.

7. A method for screening a material for preventing or treating a stress-related disease, the method comprising:
(a) treating cells expressing a disrupted in schizophrenia 1 (DISC1) protein or a gene encoding the DISC1 protein with a candidate material in vitro;
(b) measuring an expression level or activity of the DISC1 protein in the cells; and
(c) selecting, as a material for preventing or treating a stress-related disease, a material that increases the expression level or activity of the DISC1 protein as compared to a group that is not treated with the candidate material.

8. The method of claim 7, wherein the cells comprise neurons.

9. The method according to claim 7, wherein the candidate material is selected from the group consisting of a compound, a microorganism culture or extract, a natural extract, a nucleic acid, and a peptide.

10. The method according to claim 9, wherein the nucleic acid is selected from the group consisting of siRNA, shRNA, microRNA, antisense RNA, an aptamer, a locked nucleic acid (LNA), a peptide nucleic acid (PNA), and a morpholino.

11. The method according to claim 7, wherein in the measuring, the expression level is measured using one or more methods selected from the group consisting of western blotting, radioimmunoassay (RIA), radioimmunodiffusion, enzyme-linked immunosorbent assay (ELISA), immunoprecipitation, flow cytometry, immunofluorescence, Ouchterlony double immunodiffusion, a complement fixation assay, and a protein chip.

12. The method according to claim 7, wherein in the measuring, the activity is measured by measuring a degree to which the DISC1 protein decreases endoplasmic reticulum-mitochondria Ca²⁺ transfer by competitively inhibiting binding of IP₃ to IP₃R1 by binding to the IP₃R1 at the MAM.
